(19) United States
(12) Patent Application Publication
(10) Pub. No.: US 2023/0346820 A1
(43) Pub. Date: Nov. 2, 2023

(54) METHODS AND COMPOSITIONS FOR THE TREATMENT OF SARS-COV-2

(71) Applicant: THE SCRIPPS RESEARCH INSTITUTE, La Jolla, CA (US)

(72) Inventors: Malina A. BAKOWSKI, La Jolla, CA (US); Nathan BEUTLER, San Diego, CA (US); Dennis R. BURTON, La Jolla, CA (US); Arnab K. CHATTERJEE, San Diego, CA (US); Case W. MCNAMARA, San Marcos, CA (US); Thomas F. ROGERS, Del Mar, CA (US); Peter G. SCHULTZ, La Jolla, CA (US); Karen WOLFF, Encinitas, CA (US); Laura RIVA, San Diego, CA (US)

(21) Appl. No.: 17/923,171

(22) PCT Filed: Apr. 15, 2021
(86) PCT No.: PCT/US21/27535

§ 371 Date: Nov. 3, 2022

Related U.S. Application Data
(60) Provisional application No. 63/107,893, filed on Oct. 30, 2020.
(60) Provisional application No. 62/705,288, filed on Jun. 19, 2020.
(60) Provisional application No. 62/704,340, filed on May 5, 2020.

(51) Int. Cl. A61K 31/7076; A61K 31/422; A61K 31/353; A61K 31/55; A61K 31/407; A61K 31/5025; A61K 31/4725; A61K 31/4439; A61K 31/403; A61K 31/519; A61K 31/573; A61P 31/14

(52) U.S. Cl. CPC A61K 31/7076; A61K 31/422; A61K 31/353; A61K 31/55; A61K 31/407; A61K 31/5025; A61K 31/4725; A61K 31/4439; A61K 31/403; A61K 31/519; A61K 31/573; A61P 31/14

(57) ABSTRACT

Disclosed herein ar methods that are useful for treating a subject who has a pathogenic infection, such as severe acute respiratory syndrome coronavirus 2 (SARS-CoV-2); reducing the likelihood of the subject from being infected by a pathogen; and for reducing the transmission of a pathogen from a subject to others. The methods utilize a compound disclosed in Table 1 or Table 4 herein, optionally in combination with an additional agent such as an anti-infective agent.

This application claims the benefit of priority to U.S. Provisional Patent Applications No. 62/704,340 filed on May 5, 2020, No. 62/705,288 filed on Jun. 19, 2020, and No. 63/107,893 filed on Oct. 30, 2020, the disclosure of which applications are incorporated in the present disclosure as if fully set forth herein.

### BACKGROUND

Coronavirus infections can result in substantial morbidity and death. Although vaccination in general can be effective against some viral infections, vaccines are not always fully effective against certain viruses. Long term effectiveness of currently known vaccines against severe acute respiratory syndrome coronavirus 2 (SARS-CoV-2) has yet to be determined, especially in view of the emergence of SARS-CoV-2 strains that could diminish the overall impact of the vaccines. Treatment of this virus, and preventing its transmission to others, has therefore gained special importance amongst some young, elderly, or immunocompromised populations.

### SUMMARY

Thus, in one embodiment, the present disclosure provides a method for treating a subject having an infection by a pathogen. The method comprises administering to the subject a therapeutically effective amount of at least one compound selected from Table 1 or Table 4 as described herein, excluding apilimod, remdesivir, and hydroxychloroquine.

In various embodiments, the pathogen is a coronavirus. For example, in an embodiment, the coronavirus is severe acute respiratory syndrome coronavirus 2 (SARS-CoV-2).

In some embodiments, the method further comprises administering to the subject an anti-infective agent. The anti-infective agent, in various embodiments, is an anti-viral agent, such as one selected from the group consisting of entry-inhibiting drugs, uncoating inhibiting drugs, reverse transcriptase inhibiting drugs, antisense drugs, ribozyme drugs, protease inhibitors, assembly inhibiting drugs, and release inhibiting drugs. In some embodiments, the anti-viral agent comprises remdesivir.

Another embodiment of the present disclosure is the compound according to formula RFM-011-200-5 or a pharmaceutically acceptable salt thereof:

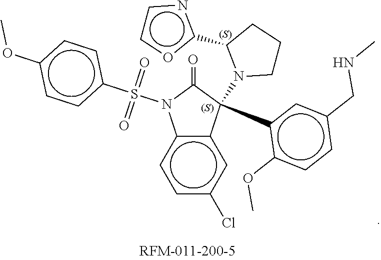

Still another embodiment of the present disclosure is the compound according to formula RFM-007-454-4 or a pharmaceutically acceptable salt thereof:

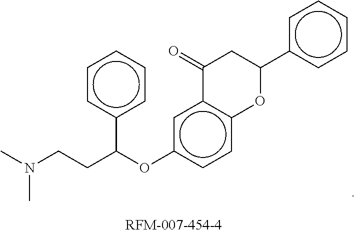

In additional embodiments, the disclosure provides a pharmaceutical composition comprising a therapeutically effective amount of at least one compound selected from Table 1 or Table 4 as described herein, a therapeutically effective amount of an anti-infective agent as described herein, and a pharmaceutically acceptable carrier.

### BRIEF DESCRIPTION OF THE DRAWINGS

FIG. 1: A primary cell-based HCI assay identifies compounds active against SARS-CoV-2 infection. A) Simplified assay workflow. B) Representative images from dimethyl sulfoxide (DMSO)-, remdesivir- or apilimod-treated wells. The entire imaged area per well (4 fields of view taken with a 10× objective and stitched together) is shown for each treatment, as well as an 8-fold magnified segment demarcated with a white box. DNA signal [4′,6-diamidino-2-phenylindole (DAPI)] is colored green, and the virus visualized with immunofluorescence is colored magenta. Infected (arrow) and uninfected (arrowhead) cells are indicated; 500 μm and 50 μm scale bars are shown in the composite and magnified images, respectively. Raw and normalized (Norm.) values calculated from the images is shown. C) Box and whiskers plot of SARS-CoV-2 assay control EC₅₀s obtained from independent biological experiments with mean indicated with a bar and all data points shown. Whiskers indicate minimums and maximums. D) Heat map images of normalized data from 1.9 μM ReFRAME screening plates. Normalized activity values for % infected cells and total cell numbers are indicated according to the scale bar and density plot for compound and control wells is shown. DMSO-treated wells are in column 24 and positive control-treated wells (blocks of wells with 2.5 μM remdesivir, 2.5 μM apilimod, or 9.6 μM puromycin) in column 23. Density plots representing the frequency of values associated with each well type are shown on the right. E) Distribution of 1.9 μM ReFRAME screen data for compound and control wells. F) Screen hit selection thresholds.

FIG. 2. Potent and selective compounds with anti-SARS-CoV-2 activity are identified in the ReFRAME library. A) The composition of the ReFRAME repurposing library with respect to clinical stage of development and disease indication. B) Dose response reconfirmation results, with the SARS-CoV-2 EC₅₀ of each compound plotted against its host cell toxicity CC₅₀ as assessed in uninfected HeLa-ACE2 cells. Dotted lines represent maximal concentrations tested in dose-response studies for the assay compounds (40 μM) and controls apilimod and remdesivir (10 μM). Activities of controls (black diamonds) and assay compounds (pink diamonds) are shown. Activity of the ReFRAME library copy of puromycin that was screened as part of this hit reconfirmation is also indicated (red diamond). C) SARS-CoV-2 EC₅₀ (blue), infected HeLa-ACE2 EC₅₀ (orange) and uninfected HeLa-ACE2 CC₅₀ dose response curves for the remdesivir, apilimod and puromycin control compounds ran as part of ReFRAME hit reconfirmation. D) Classification of 75 potent and selective and 135 weakly active or non-selective compounds according to their functional annotation. E) A representative output of the synergy analysis for apilimod plus remdesivir drug interaction landscape (single replicate), showing an overall additive effect (−10<δ<10). F) Additive response between remdesivir and two fixed concentrations of apilimod (approximately EC₃₀ and EC₆₅ during the synergy experiment). Medians+/−sem of technical triplicates are shown.

FIG. 3. Effects of MOI on control compound EC₅₀s. Activity of remdesivir, apilimod, and puromycin controls in the SARS-CoV-2/HeLa-ACE2 assay was assessed with MOIs ranging from 1 to 26. EC₅₀ of each compound at the indicated MOI is shown.

### DETAILED DESCRIPTION

The present disclosure relates, in part, to methods of treating a subject who has a pathogenic infection, such as SARS-CoV-2. In early December of 2019, the severe acute respiratory syndrome coronavirus 2 (SARS-CoV-2) was identified as the cause of rapidly increasing numbers of severe pneumonia-like symptoms termed COVID-19 (Huang, C. et al. Clinical features of patients infected with 2019 novel coronavirus in Wuhan, China. Lancet 395, 497-506, doi:10.1016/S0140-6736(20) 30183-5 (2020)). Since then, SARS-CoV-2 has rightfully been given its pandemic status by the World Health Organization (WHO). As of Feb. 10, 2021 SARS-CoV-2 has spread throughout the world causing more than 106,555,200 confirmed infections and more than 2,333,440 reported deaths in 223 different countries (Organization, W. H. Vol. 2020 (ed World Health Organization) (World Health Organization, 2020)). Development of several effective anti-SARS-CoV-2 vaccines will no doubt contribute to the control of the pandemic, however emergence of SARS-CoV-2 strains with escape mutations that render some of the vaccines less effective and overall limited global supply of COVID-19 vaccines make a case for continued effort to identify therapeutic interventions. Yet, despite an extensive effort by the research community, antiviral treatment options for COVID-19 remain extremely limited. These include corticosteroids such as dexamethasone (Group, R. C. et al. Dexamethasone in Hospitalized Patients with Covid-N Engl J Med,
doi:10.1056/NEJMoa2021436 (2020) and the intravenously-delivered antiviral remdesivir (de Wit, E. et al. Prophylactic and therapeutic remdesivir (GS-5734) treatment in the rhesus macaque model of MERS-CoV infection. Proc Natl Acad Sci USA 117, 6771-6776,
doi:10.1073/pnas.1922083117 (2020); Sheahan, T. P. et al. Broad-spectrum antiviral GS-5734 inhibits both epidemic and zoonotic coronaviruses. Sci Transl Med 9,
doi:10.1126/scitranslmed.aal3653 (2017); Lo, M. K. et al. GS-5734 and its parent nucleoside analog inhibit Filo-, Pneumo-, and Paramyxoviruses. Sci Rep 7, 43395, doi:10.1038/srep43395 (2017)) for treatment of patients with severe or critical COVID-19. Remdesivir, a nucleotide analog prodrug and an RdRp inhibitor with broad antiviral activity demonstrated positive clinical endpoints in a Phase III Adaptive COVID-19 Treatment Trial (median time to recovery shortened from 15 to 11 days; Health, N. I. o. (2020) that justified its emergency use authorization by the US Food & Drug Administration for treatment of hospitalized COVID-19 patients (Administration, U. S. F. D. (ed U.S. Food & Drug Administration) (U.S. Food & Drug Administration, 2020). However, it, together with hydroxychloroquine, lopinavir and interferon regimens has recently failed to reduce mortality of hospitalized COVID-19 patients in a large multi-center WHO SOLIDARITY trial (Pan, H. et al. Repurposed antiviral drugs for COVID-19—interim WHO SOLIDARITY trial results. medRxiv, 2020.2010.2015.20209817,
doi:10.1101/2020.10.15.20209817 (2020)). The present disclosure also relates in part to screens of a large drug library (ReFRAME) in two different cell-based SARS-CoV-2 infection assays and in a remdesivir potentiation format, and the profiling of the identified hits in secondary orthogonal assays. This screening cascade and subsequent hit prioritization identified and validated a promising hit, MK-4482, as a potent inhibitor of SARS-CoV-2, in vitro findings which translated to an in vivo hamster model of SARS-CoV-2 infection. Other hits identified in these studies are useful for repurposing into therapies and tools for elucidating coronavirus replication pathways.

### Definitions

As used herein, and unless otherwise specified to the contrary, the term “compound” is inclusive in that it encompasses a compound or a pharmaceutically acceptable salt, stereoisomer, and/or tautomer thereof. Thus, for instance, a compound as described herein includes a pharmaceutically acceptable salt of a tautomer of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing the spread or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Screening Assay and Methods

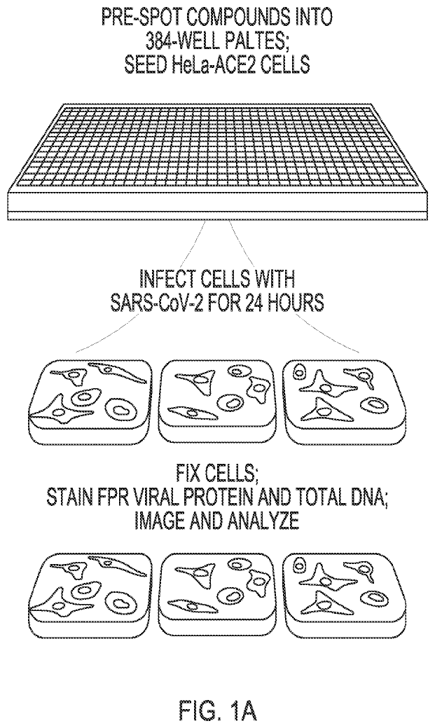

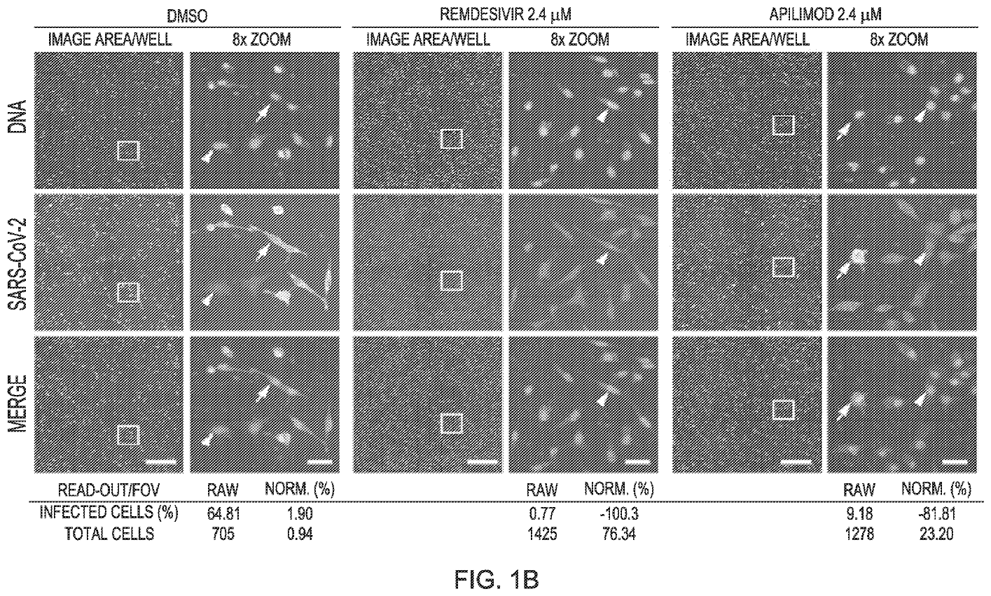

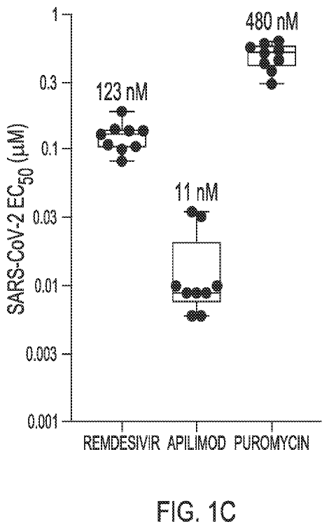

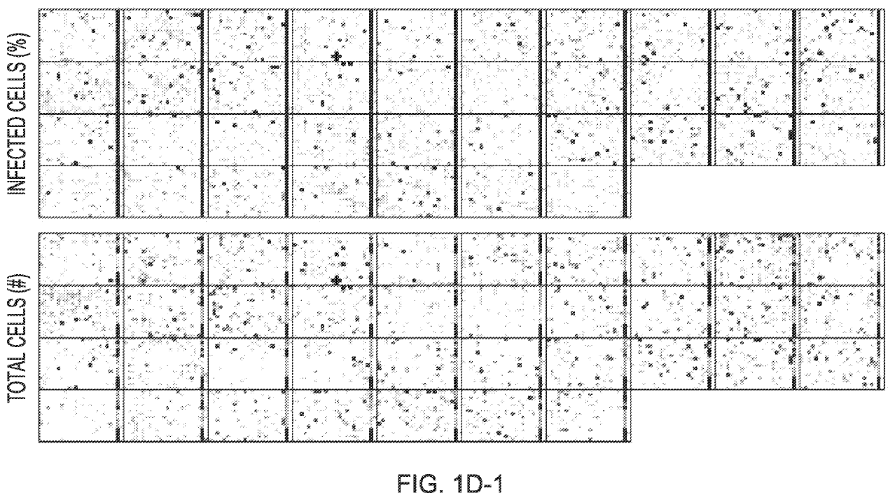

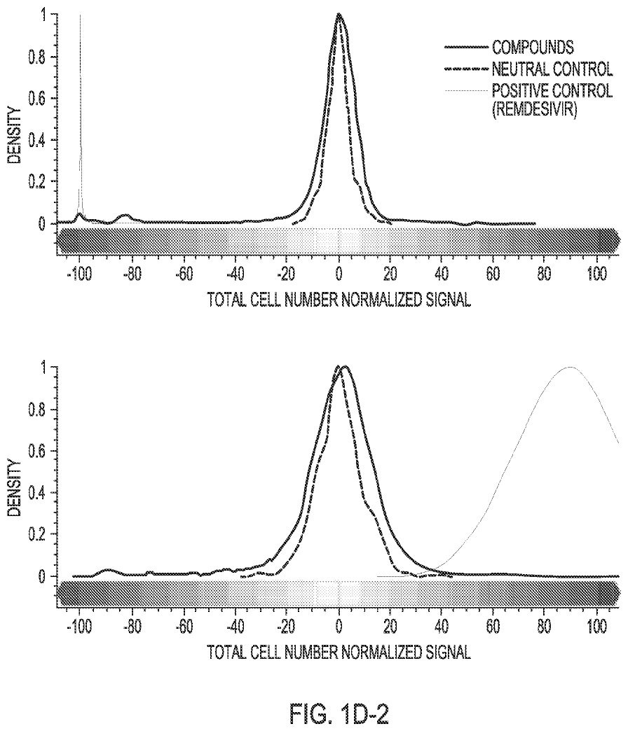

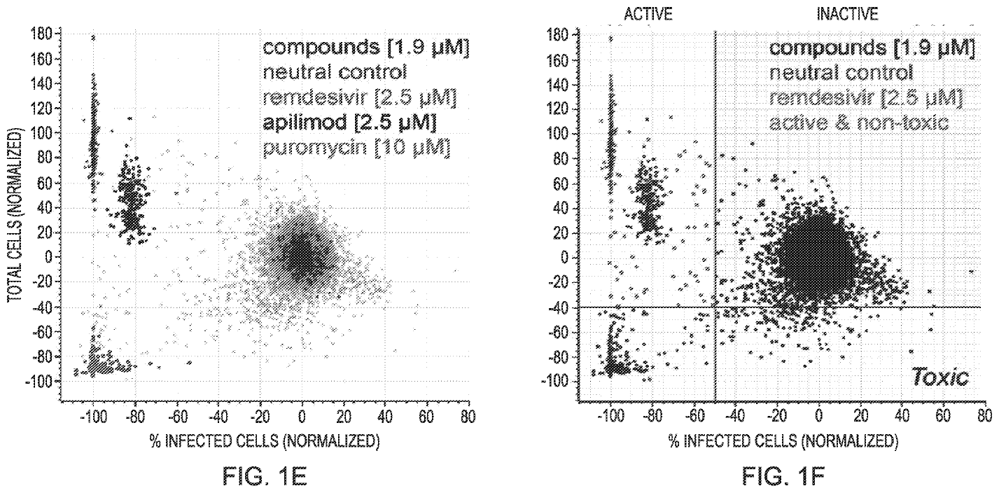

The ReFRAME (Repurposing, Focused Rescue, and Accelerated Medchem) drug collection is an extensive drug repurposing library containing nearly 12,000 small-molecule drugs shown to be appropriate for direct use in humans (5) and provides a rich resource to discover new treatments that can be useful as additional monotherapies or in combination with remdesivir to further enhance efficacy and reduce drug resistance potential. To identify compounds in this library that could inhibit entry or replication of SARS-CoV-2 in human cells, we developed a high-content imaging (HCI) 384-well format assay using HeLa cells expressing the human SARS-CoV-2 receptor, the angiotensin-converting enzyme 2, or ACE2 (HeLa-ACE2). In this assay, HeLa-ACE2 cells are infected with SARS-CoV-2 virus in the presence of compounds of interest, and viral infection is quantified 24 hours later (FIG. 1, panel A). The assay relies on immunofluorescent detection of SARS-CoV-2 proteins with sera that is purified from patients exposed to the virus, which together with host cell nuclear staining allows for quantification of the percent infected cells in each well (FIG. 1, panel B).

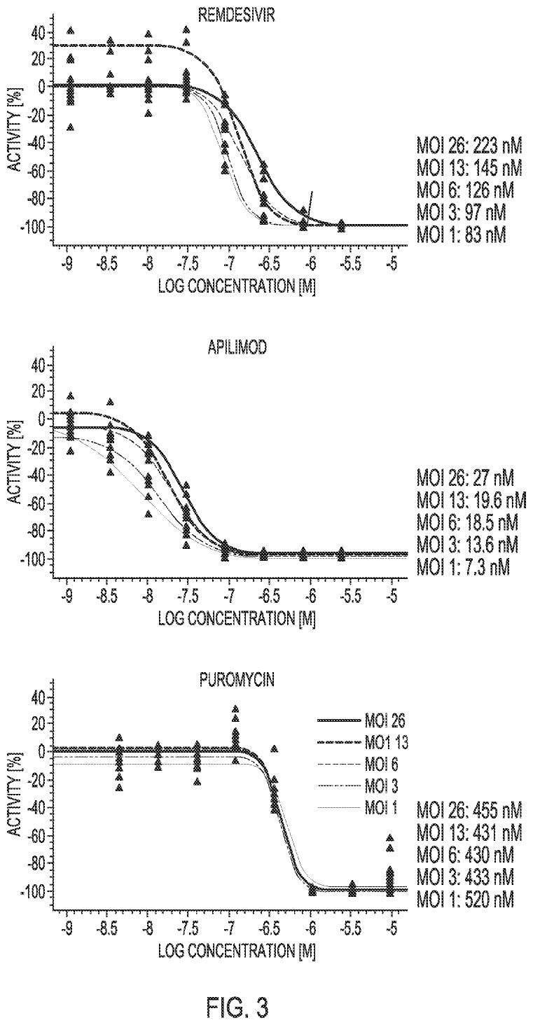

We validated the assay using compounds with reported activity against Ebola and suspected or previously verified activity against SARS-CoV-2: remdesivir (GS-5734) (6) (EC₅₀=194±20 nM; average±sem of 5 independent experiments) and the PIKfyve inhibitor apilimod (EC₅₀=50 f 11 nM, average±sem of 4 independent experiments) (FIG. 1, panel B). Remdesivir at elevated concentrations was able to eliminate infected cells almost completely (FIG. 1, panel C) and we used it at a concentration of 2.5 μM as a positive control, with data normalized to it and neutral DMSO control wells. While apilimod was more potent than remdesivir, it had a fractionally lower maximal efficacy (85-90% of uninfected cells at the highest effective concentrations) compared to remdesivir. Additionally, we assessed compound toxicity in the context of infection by quantifying the total cell numbers per well, with cytotoxic protein synthesis inhibitor puromycin as a positive control (average EC₅₀=547±27 nM, average±sem of 5 independent experiments; HeLa-ACE2 CC₅₀=2.45±0.23 μM, average±sem of 5 independent experiments). Notably, a concomitant increase in cell numbers coincided with the antiviral activity of remdesivir and apilimod, likely due to reduction in proliferation of infected cells (FIG. 1, panels B-E). Altering the multiplicity of infection had modest effects on the potency of control compounds in the same experiment, with a 2.7-fold increase in remdesivir's EC₅₀ from MOI=1 to MOI=26, and a 3.7-fold increase in the EC₅₀ of apilimod, but not that of puromycin (FIG. 3).

Using the developed assay, we ran a pilot screen to assess the activity of 148 small molecules with suspected therapeutic potential against coronavirus infections (7) (RZ′=0.84). We identified 19 compounds with an EC₅₀<9.6 μM and, based on data obtained from an uninfected HeLa-ACE2 24-hour live/dead assay, 10 of these were selective (uninfected HeLa-ACE2 CC₅₀/SARS-CoV-2 EC₅₀>10 or uninfected HeLa-ACE2 CC₅₀>40 μM) (Table 1). This included library/screening lots of apilimod (EC₅₀=184 nM, CC₅₀>40 μM) and remdesivir (EC₅₀=300 nM CC₅₀>40 μM) that were “rediscovered” in the assay. The higher EC₅₀ of apilimod and remdesivir are likely due to slight degradation over time in the screening deck compared to freshly prepared control powder stock.

TABLE 1
Screen of ReFRAME library, anti-viral activities, and synergy with remdesivir

| | | | | | | CC₅₀ |
|---|---|---|---|---|---|---|
| | | | | | | [μM] |
| | | | | | | (Geo- |
| | | | | | HeLa- | means, |
| | | Synergy | | CoV-2 | ACE-2 | n ≥ 2 |
| | | score | HeLa- | EC₅₀ | EC₅₀ | except |
| | | with | ACE2 | [μM] | [μM] | where |
| | | remde- | CC₅₀/ | (Geo- | (Geo- | indi- |
| | | sivir | CoV-2 | means, | means, | cated: |
| Name | Structure | (δ) | EC₅₀ | n ≥ 2) | n ≥ 2) | *n = 1) |
| RFM-011- 200-5 | 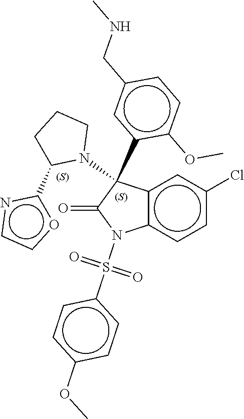 | — | >4.37 | 1.93 | 4.24 | >8.46 |
| | LCMS mass found M + H = 623.6 | | | | | |
| RFM-007- 454-4 | 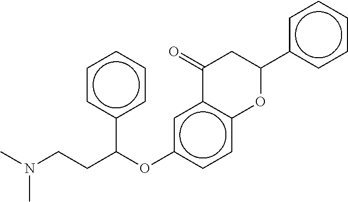 | — | >6.13 | 3.83 | >9.59 | >23.47 |
| | LCMS mass found M + H = 402.2 | | | | | |
| Remdesivir | 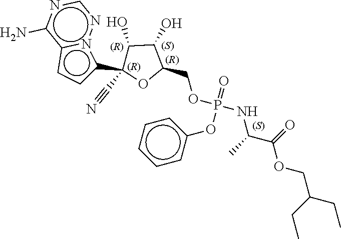 | n/a | 78.7 | 0.127 | >9.597 | 9.973 |
| Apilimod | 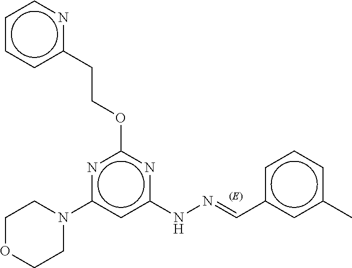 | 3.568 | >4353.33 | <0.006 | >9.602 | >27.804 |
| MK-4482/ EIDD-2801/ Molnupiravir | 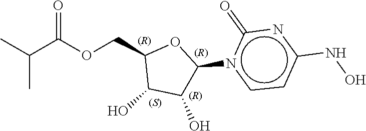 | — | >4.21 | >9.454 | >9.59 | >39.813 |
| N- hydroxy- cytidine | 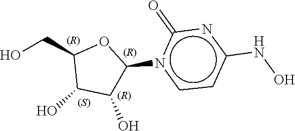 | — | >18.13 | 2.069 | >9.595 | >37.514 |
| Asenapine maleate | 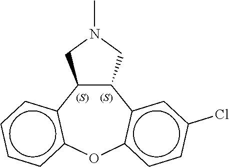 | — | > 14.68 | >2.427 | >9.6 | >35.637 |
| manidipine | 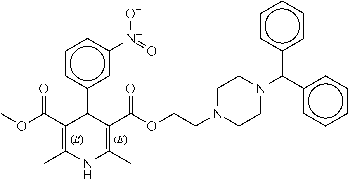 | −4.727 | >2.48 | >6.888 | >9.596 | >17.068 |
| Trimipramine | 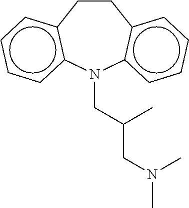 | — | 10.11 | 2.106 | >9.59 | 21.282 |
| Hanfangchin A | 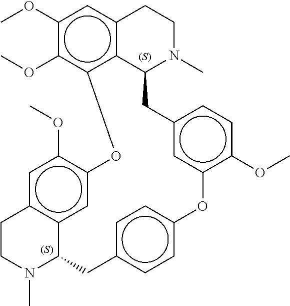 | −3.61 | >16.08 | 1.177 | >9.205 | >18.921 |
| Dabigatra- netexilate mesilate | 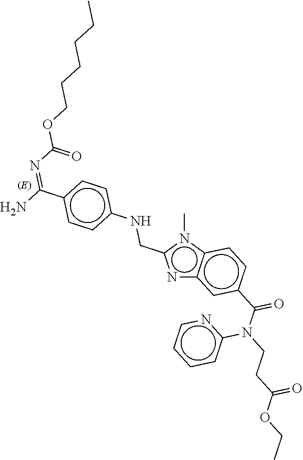 | — | >6.50 | 4.083 | >9.59 | >26.539 |
| Boceprevir | 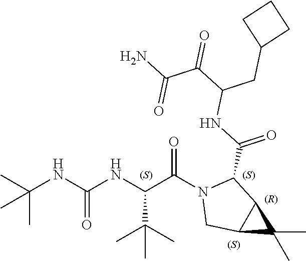 | — | 4.27 | >9.34 | >7.08 | >39.83 |
| nebivolol hydrochloride | 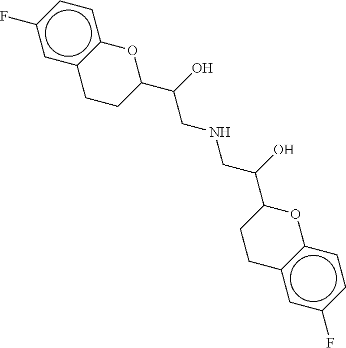 | — | >3.75 | 2.962 | >9.116 | >11.107 |
| Sorafenib | 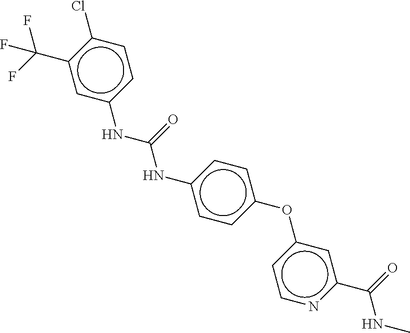 | — | >1.52 | >5.675 | >8.306 | >8.647 |
| Cepharanthine | 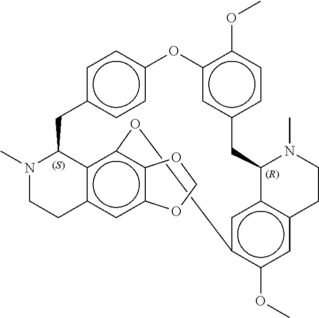 | — | >25.76 | 0.988 | >9.59 | >25.447 |
| raloxifene hydrochloride | 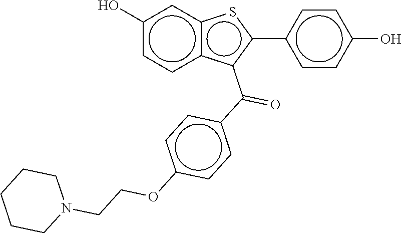 | — | >2.97 | 4.476 | >8.714 | >13.281 |
| hydroxy- chloro- quine, nasal | 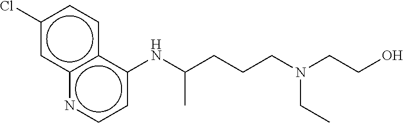 | −1.572 | >40.04 | 0.458 | >5.883 | >18.345 |
| AZD-5363 | 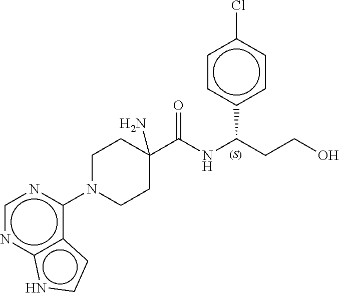 | — | 8.61 | >4.63 | >3.39 | >39.83 |
| Ponatinib | 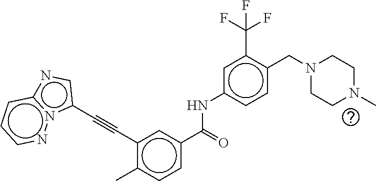 | — | >3.18 | 1.608 | 1.246 | >5.113 |
| MK-2206 | 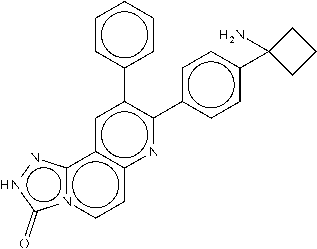 | 2.087 | >20.63 | 1.103 | >9.59 | >22.753 |
| Ralimetinib mesylate/ LY2228820 | 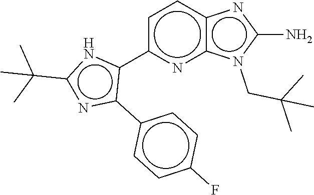 | −0.846 | 14.6 | 1.874 | >6.867 | 27.356 |
| Reserpine | 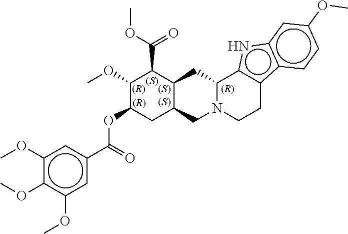 | — | >6.60 | >5.515 | >9.6 | >36.371 |
| amiodarone | 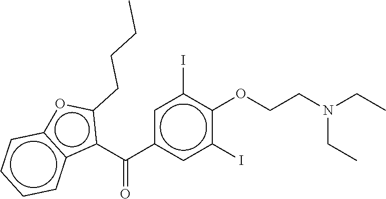 | 0.683 | 15.52 | 1.027 | >9.59 | 15.937 |
| Thiopro- perazine | 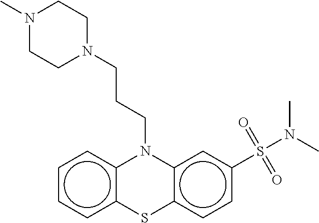 | — | 7.29 | 3.031 | >9.59 | 22.105 |
| Digoxin | 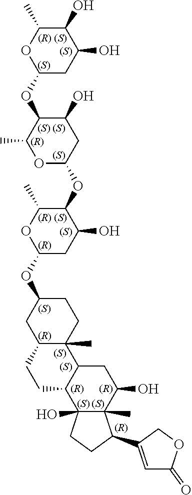 | — | 0.72 | 0.166 | 0.325 | 0.12 |
| BGB324/ R-428 | 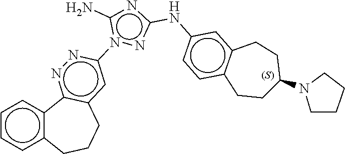 | 7.223 | 13.05 | 1.202 | >9.151 | 15.684 |
| nelfinavir mesylate | 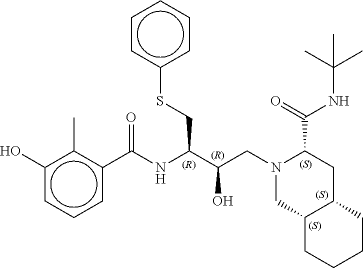 | −1.233 | >2.08 | >8.643 | >9.595 | >18.017 |
| TILORINE DIHYDRO- CHLORIDE | 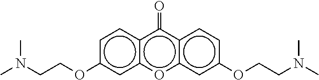 | — | 11.07 | 0.884 | 7.464 | 9.787 |
| Ozanimod | 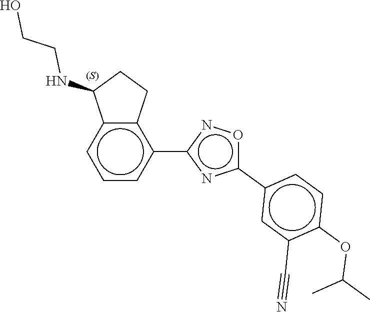 | −4.622 | >5.60 | 2.634 | >8.738 | >14.744 |
| APY0201 | 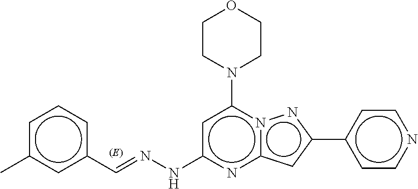 | — | >2853.95 | 0.014 | >9.59 | >39.405 |
| GW-803430 | 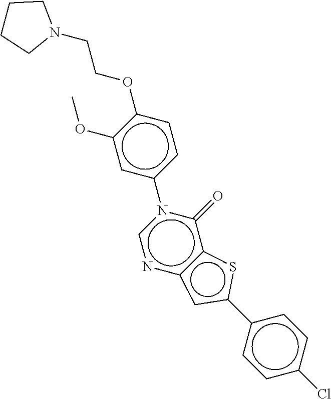 | — | 11.16 | 1.696 | >7.876 | 18.919 |
| ABT-239 | 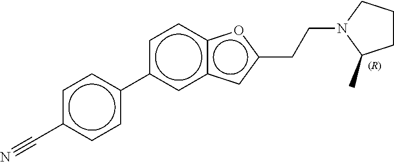 | — | 11.18 | >3.33 | >7.49 | >37.25 |
| pyronaridine | 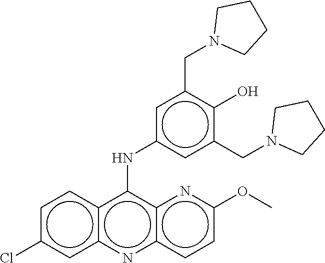 | — | >4.86 | 1.523 | 3.409 | >7.393 |
| R-7112 | 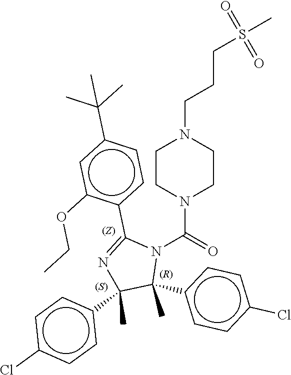 | — | >4.61 | 3.23 | >9.60 | >14.89 |
| diclofensine | 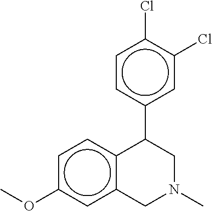 | — | <1.28 | >7.891 | >9.59 | 10.082 |
| Simeprevir | 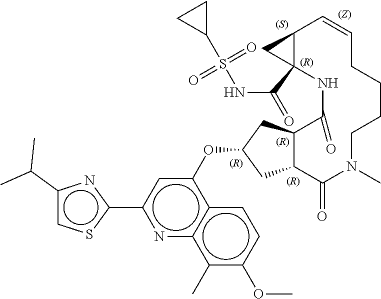 | −0.667 | >2.81 | >7.884 | >9.472 | >22.165 |
| OCTO- CLOTHEPIN analog | 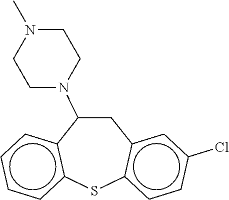 | — | >11.42 | 2.49 | >8.73 | >28.40 |
| Thalicarpine Analog | 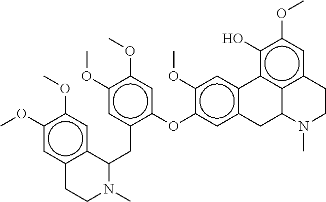 | — | 26.77 | >0.80 | >8.78 | >21.50 |
| SAX-187 | 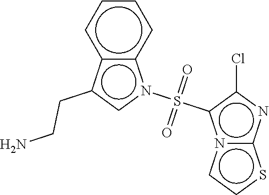 | — | 4.02 | 1.846 | >9.365 | 7.428 |
| MP-412 | 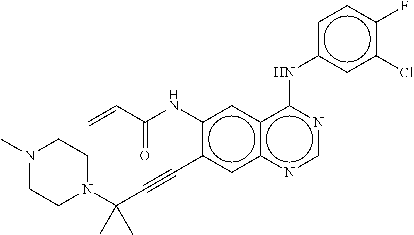 | — | 4.18 | 0.761 | 1.248 | 3.181 |
| 8-Chloro- adenosine | 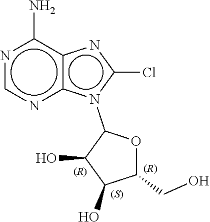 | — | >12.80 | 0.927 | >9.59 | >11.861 |
| DESMETH- YLASTEMI- ZOLE | 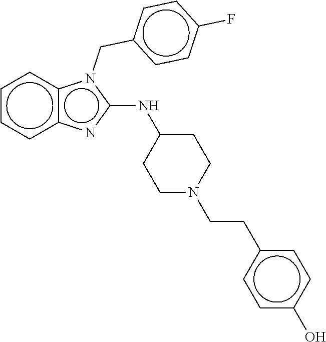 | — | 6.81 | 2.375 | >9.59 | 16.166 |
| AHR-5333 | 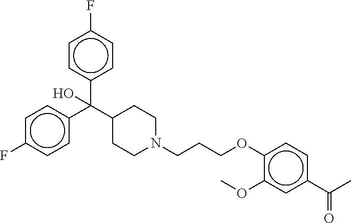 | — | 5.44 | >4.53 | >7.10 | >24.65 |
| Osimertinib | 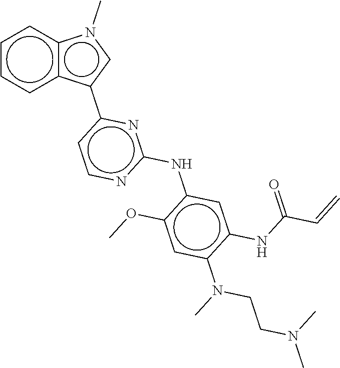 | −0.722 | >18.79 | 0.962 | >4.226 | >18.07 |
| PIPAMA- ZINE | 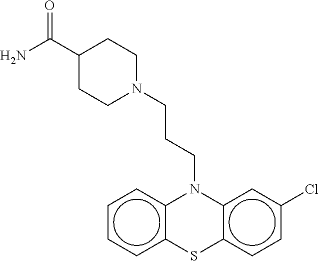 | 1.112 | >5.88 | 3.408 | >9.59 | >20.056 |
| AQ-13 | 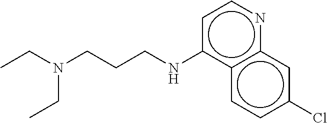 | −1.765 | 32.34 | 0.8 | >9.598 | 25.891 |
| YM-75440 | 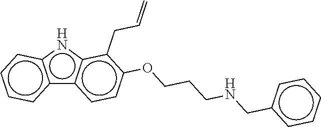 | — | 4.89 | 1.638 | >6.579 | 8.005 |
| Alkene Stereoisomer analog of Rilapine | 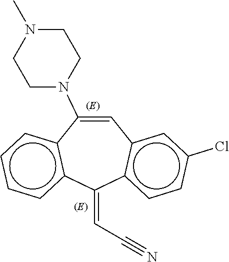 | — | >13.32 | 2.99 | >9.59 | >39.83 |
| Analog of Centbucridine | 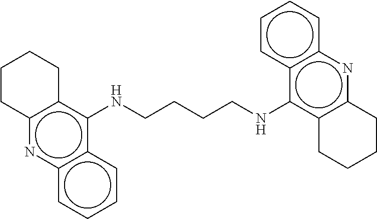 | — | 18.26 | 0.44 | 4.26 | 8.1 |
| tesevatinib, | 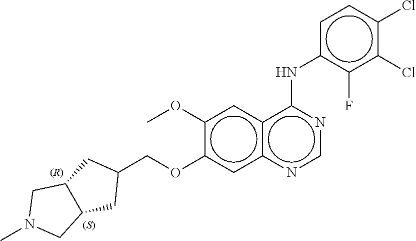 | — | >8.46 | 2.435 | >8.163 | >20.599 |
| Ferroquine | 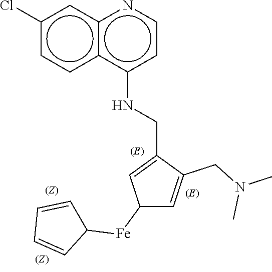 | −2.076 | 10.29 | 1.306 | >6.801 | 13.437 |
| TAK-070 | 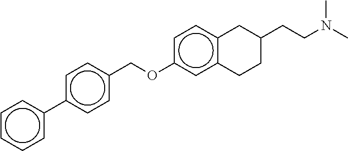 | — | 7.19 | 1.406 | >5.503 | 10.117 |
| SMN-C3 | 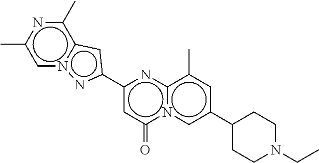 | −0.365 | 5.12 | 1.399 | >8.144 | 7.16 |
| LG-6-101 | 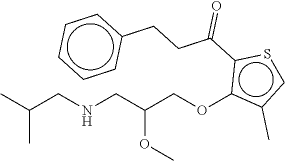 | — | 10.3 | 3.457 | >9.59 | >35.595 |
| CFI-400945 | 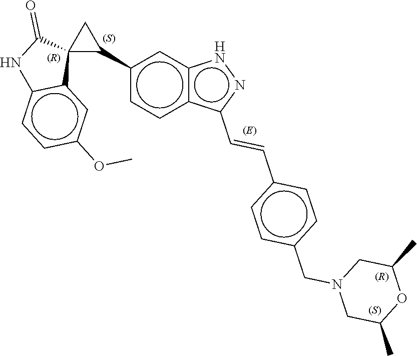 | — | <1.02 | >7.001 | >0.117 | 7.176 |
| NVX-207 | 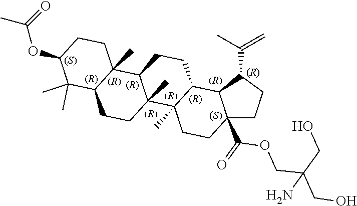 | — | 4.37 | 1.873 | 5.879 | 8.189 |
| KC 11404 | 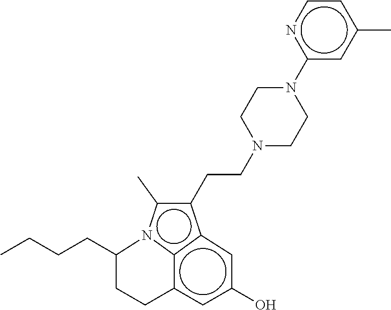 | −2.817 | >8.39 | 2.496 | >9.59 | >20.94 |
| NNC 090026 | 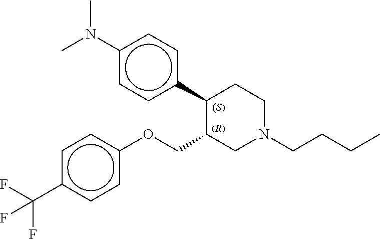 | — | >5.59 | 2.747 | >8.971 | >15.351 |
| CR-3124 | 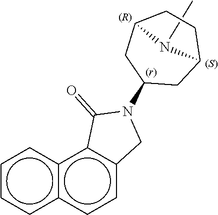 | — | >6.67 | 5.968 | >9.59 | >39.82 |
| ZUCLPEN THIXL | 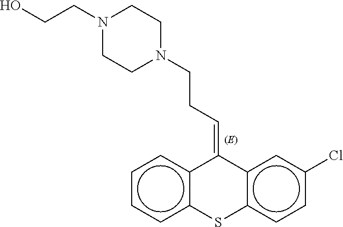 | — | >8.08 | 2.084 | >9.59 | >16.84 |
| R-116301 | 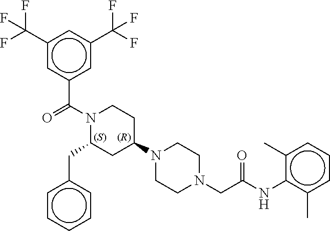 | — | 3.73 | 6.83 | >9.59 | 25.48 |
| ACOLBI- FENE | 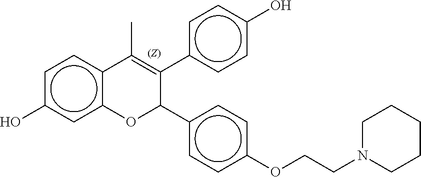 | — | 2.8 | 4.795 | >9.59 | 13.432 |
| Thalicarpine | 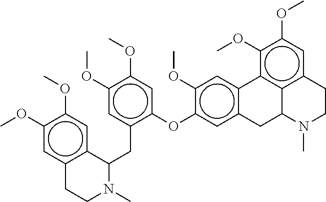 | — | >45.67 | 0.582 | >9.602 | >26.586 |
| YM 430 | 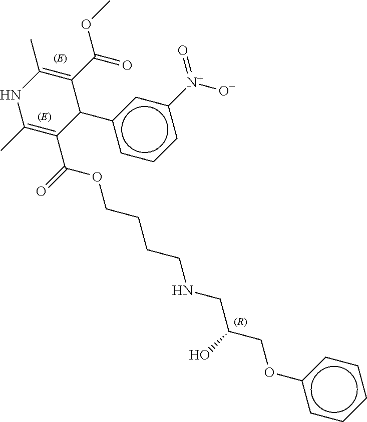 | — | 7.01 | 2.88 | >7.80 | >20.20 |
| NCO 700 | 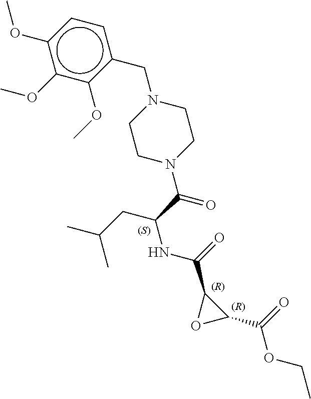 | 0.209 | >50.44 | 0.79 | >5.08 | >39.82 |
| Dutacatib | 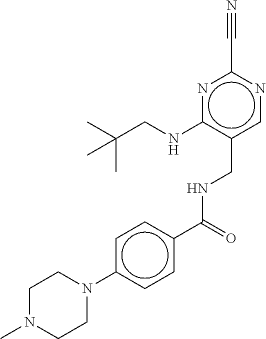 | 2.955 | >13.43 | 2.689 | >9.59 | >36.111 |
| M-55532 | 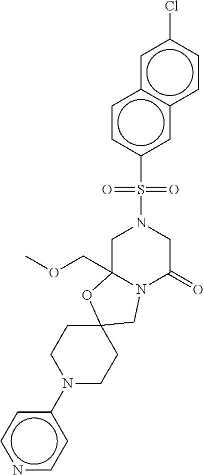 | — | 6.81 | 2.77 | >6.45 | 18.88 |
| YM 161514 | 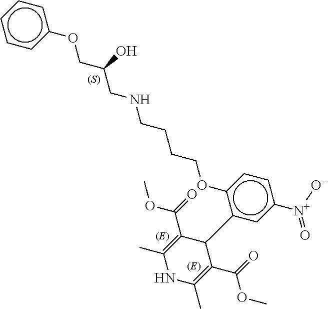 | 0.601 | 6.08 | 1.863 | >8.47 | 11.334 |
| Integrity ID 725781 | 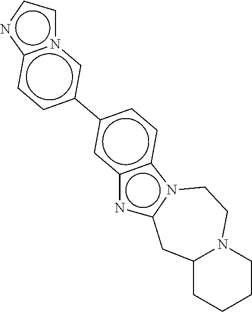 | — | 4.41 | >9.04 | >9.59 | >39.83 |
| MONATEPIL | 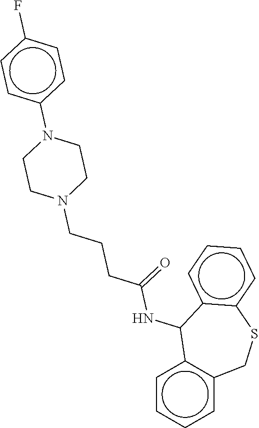 | — | >5.01 | >7.792 | >9.6 × [symbol] | >39.018 |
| Pancopride | 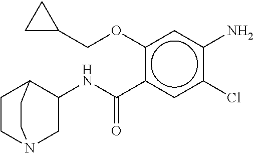 | 0.648 | 4.32 | >8.64 | >9.59 | >37.29 |
| Halofantrine HCl | 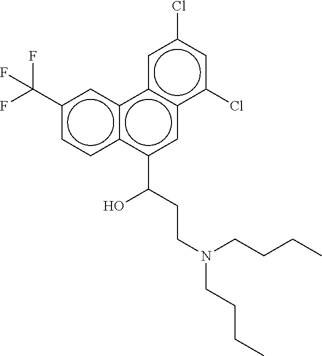 | −3.509 | >20.66 | 1.158 | >6.187 | >23.927 |
| AMOPYRO- QUINE | 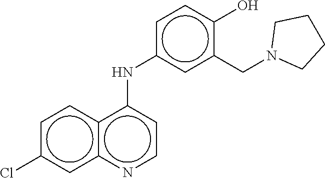 | — | 19.02 | 0.904 | >9.59 | 17.205 |
| SLV-307 | 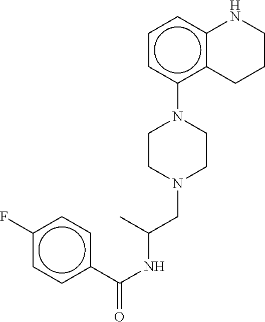 | — | 4.15 | >9.59 | >6.70 | >39.83 |
| A 81834 | 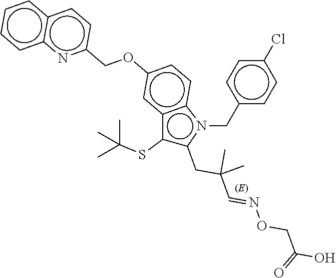 | — | >3.02 | >7.467 | >9.59 | >22.556 |
| L 796568 | 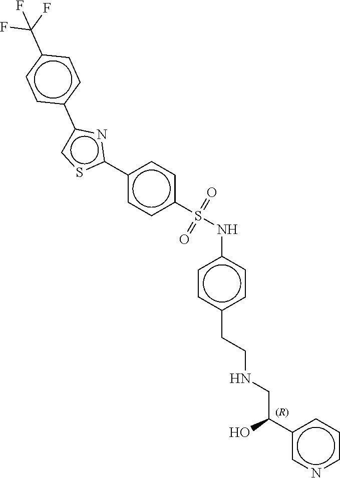 | — | 5.28 | 1.045 | 3.142 | 5.512 |
| ROPITOIN | 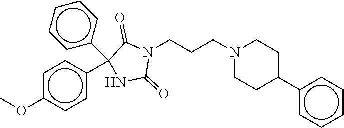 | — | 5.28 | 4.075 | >9.59 | 21.533 |
| S-33084 | 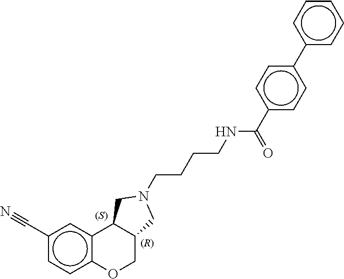 | — | >6.34 | 3.909 | >9.59 | >24.786 |
| RFM-011- 761-3 | 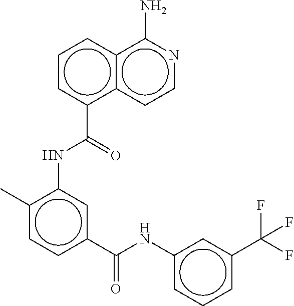 | — | >3.37 | 6.31 | >9.06 | >21.24 |
| Narasin | 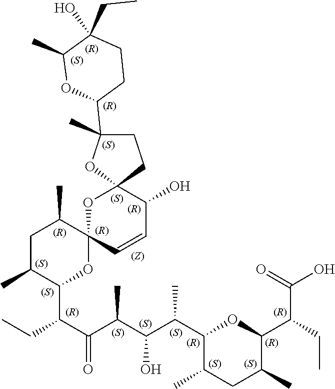 | — | 2.31 | 1.503 | 5.992 | 3.469 |
| Ethyliso- butrazine Hydro- chloride | 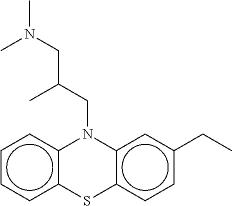 | — | 10.48 | 1.996 | >9.59 | 20.916 |
| GS-9901 | 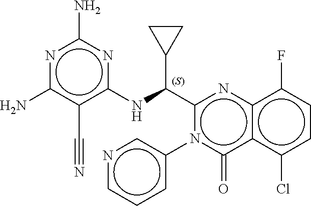 | — | >8.17 | 3.807 | >9.59 | >31.113 |
| Des-ethyl human metabolite of amodiaquine | 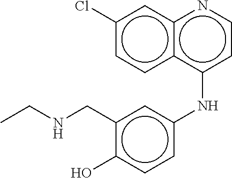 | — | >16.69 | 1.22 | >9.59 | >20.40 |
| Mequitazine | 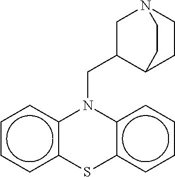 | — | 6.83 | 2.53 | >9.59 | 17.269 |
| Rupinavir | 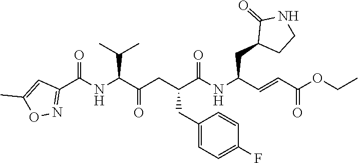 [SMILES: CCOC(=O)/C=C/[C@H](C[C@@H]1CCNC1=O)NC(=O)[C@@H](CC(=O)[C@@H](NC(=O)c1cc(C)on1)C(C)C)Cc1ccc(F)cc1] | | 4.15 | >9.59 | >9.59 | >39.84* |
| albaconazole | 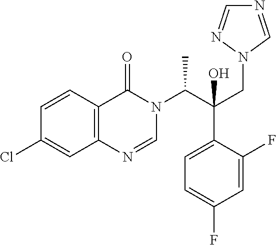 [SMILES: C[C@@H](n1cnc2cc(Cl)ccc2c1=O)[C@](O)(Cn1cncn1)c1ccc(F)cc1F] | | 6.6 | >6.04 | >9.59 | >39.84* |
| Balicatib | 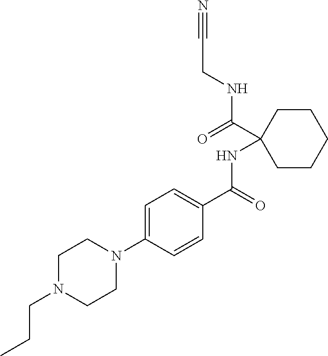 [SMILES: CCCN1CCN(c2ccc(C(=O)NC3(C(=O)NCC#N)CCCCC3)cc2)CC1] | | >7.79 | 5.11 | >9.59 | >39.84* |
| Risdiplam | 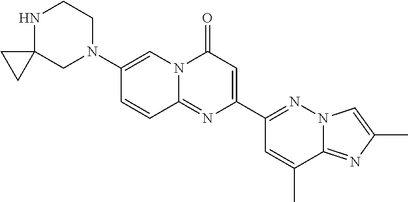 [SMILES: Cc1cn2nc(-c3cc(=O)n4cc(N5CCNC6(CC6)C5)ccc4n3)cc(C)c2n1] | | 2.53 | >7.05 | >7.05 | >17.82 |
| Ebselen | 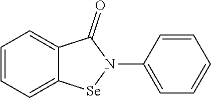 [SMILES: O=c1c2ccccc2[se]n1-c1ccccc1] | | 3.13 | >8.50 | 6.02 | >26.62 |
| Arbidol | 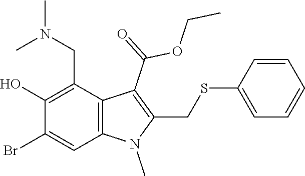 [SMILES: CCOC(=O)c1c(CSc2ccccc2)n(C)c2cc(Br)c(O)c(CN(C)C)c12] | | 4.88 | >5.97 | >9.34 | >29.12 |
| Z LVG CHN2 | 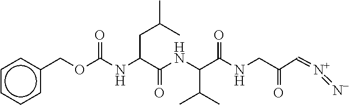 | — | >554.29 | 0.072 | >9.59 | >39.82 |
| Amodio- aquine | 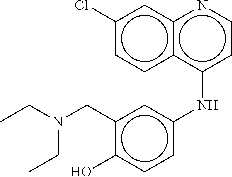 | — | >33.48 | 1.19 | >0.865 | >39.84* |
| Imatinib mesylate | 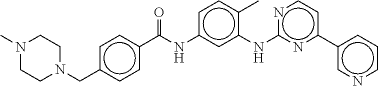 | — | >7.43 | 5.36 | >9.59 ×÷ 1 | >39.84* |

[symbol]

indicates data missing or illegible when filed

We screened the 12,000-compound ReFRAME repurposing library at final concentrations of 1.9 μM and 9.6 μM. Assay quality was maintained throughout both screens, as shown in Table 2 below (RZ′ of 0.87 and 0.72, respectively).

TABLE 2
Primary and validation screen statistics.

| | ReFRAME | ReFRAME | |
|---|---|---|---|
| Library | [1.9 μM] | [9.6 μM] | Total^{b} |
| Compounds screened | 12,983 | 12,983 | 12,983 |
| Primary hits^{a} | 64 | 280 | 326 |
| Average RZ′ | 0.8695 | 0.7241 | 0.7968 |
| Hit rate | 0.49% | 2.16% | 2.51% |
| Tested in dose response (DR) | 63 | 279 | 325 |
| EC₅₀ < 9.4 μM | 45 | 205 | 233 |
| (EC₅₀ > 9.4 μM | | | (42) |
| (weak activity)) | | | |
| Reconfirmation rate | 71.4% | 73.5% | 71.7% |
| EC₅₀ < 9.4 μM, | 10 | 49 | 53 |
| CC₅₀/EC₅₀ > 10 | | | |
| Potent and selective hits | 15.9% | 17.6% | 16.3% |

^{a}Primary hit thresholds: >50% inhibition of infection, <40% cell toxicity; 6 border-line hits included in 1.9 μM
^{b}non-overlapping hits from 1.9 and 9.6 μM screens

In addition, a clear distinction was apparent in the activity profiles of DMSO vehicle-(neutral control), remdesivir- (positive control), apilimod-, and puromycin- (toxicity control)-treated wells (FIG. 1, panels D and E). Hit selection was based on demonstration of >50% reduction in the number of infected cells per well (<−50% activity normalized to neutral controls minus inhibitors) and <40% toxicity based on the total cell number per well (>−40% activity normalized to compound activities, including 10 μM puromycin) (FIG. 1, panels E and F) identifying 61 primary hits at 1.9 μM and 266 primary hits at 9.6 μM screening concentrations (hit rates of 0.51 and 2.24%, respectively), with a total of 311 hits.

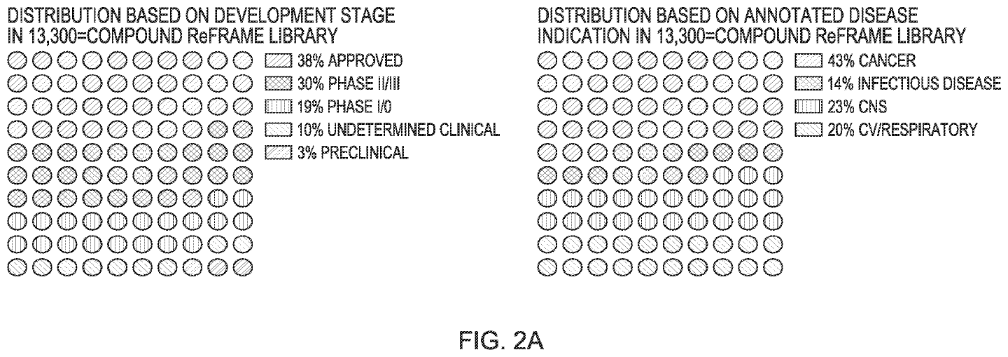

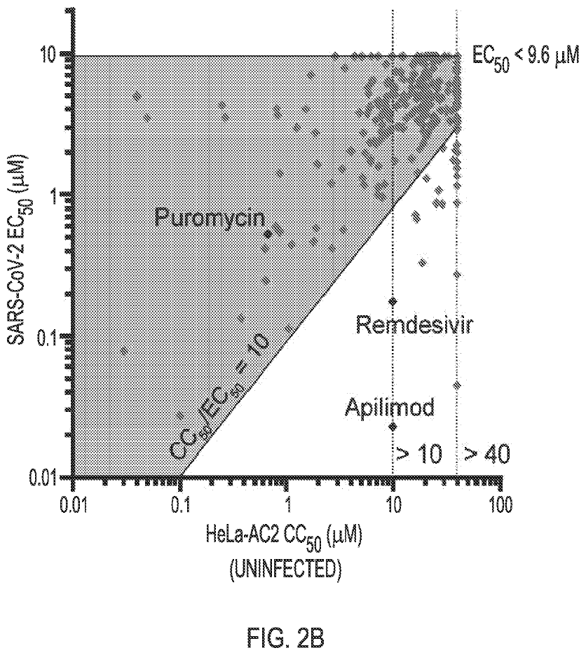

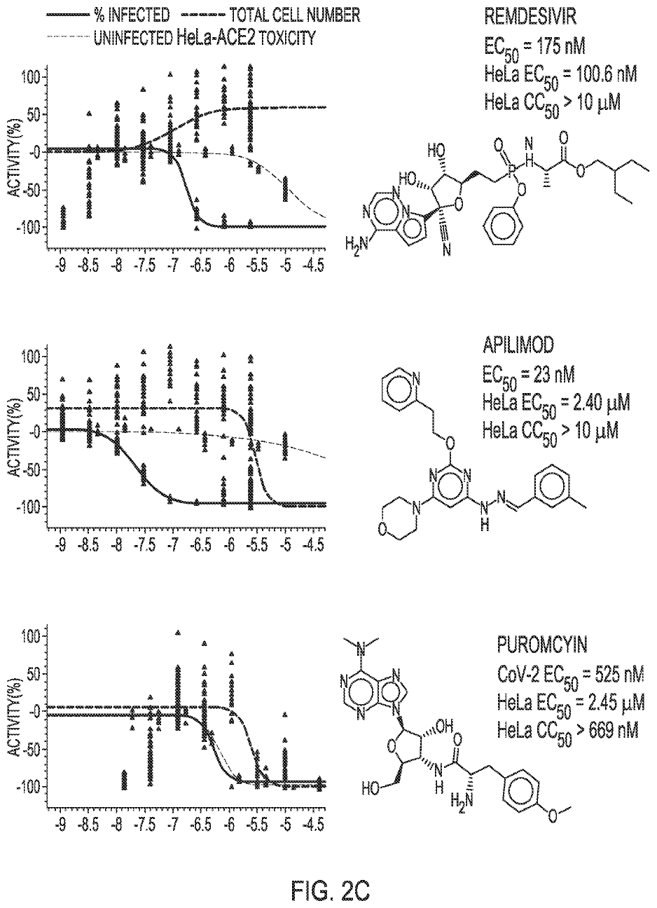

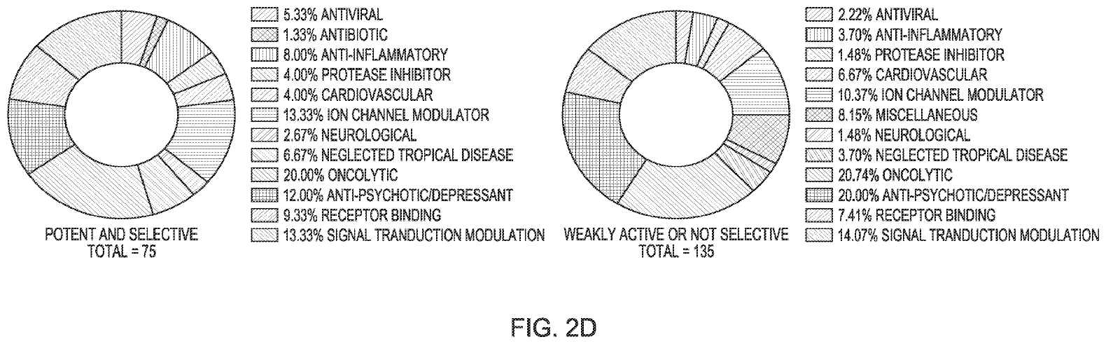

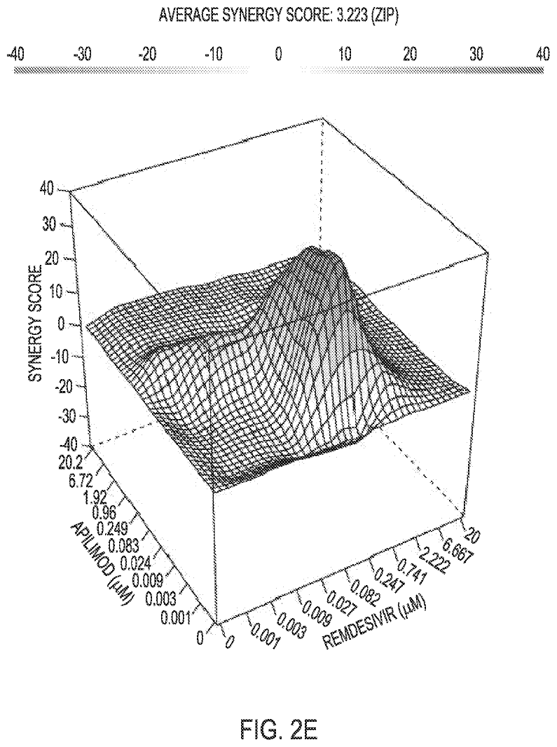

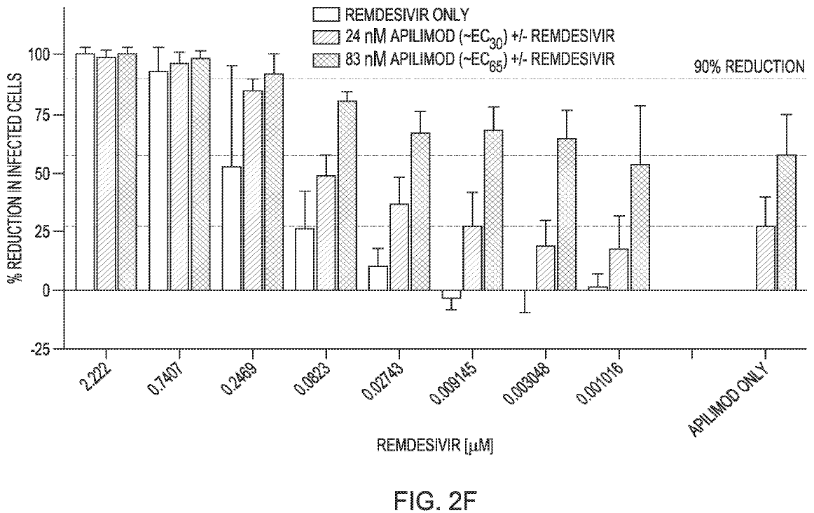

The hit rate for the primary screen of the ReFRAME library was high (2.51%), but not unexpected for this collection of bioactive small molecules, many of which are approved drugs or in clinical phases of development and used for a wide assortment of indications (FIG. 2, panel A). To reconfirm and assess potency and selectivity of the primary hits we tested 325 of the available compounds in a 10-point 1:3 dilution dose response format with a top concentration of 9.6 μM. Of these, 233 (71.7%) demonstrated activity with EC₅₀<9.6 μM against SARS-CoV-2 and an additional 42 (12.9%) showed weak activity (EC₅₀>9.6 μM). However, many of the primary screen hits were also cytotoxic, with an unacceptably low selectivity ratio as determined in uninfected HeLa-ACE2 cells (uninfected CC₅₀/EC₅₀<10) (Table 3, FIG. 2, panel B).

TABLE 3
Selected reconfirmed hits with activity and selectivity against SARS-CoV-2

| | | | | SARS | Uninfected | Synergy |
|---|---|---|---|---|---|---|
| | | | | CoV-2 | HeLa- | score with |
| Compound/ | Target/ | Clinical | PK/Exposure | EC₅₀ | ACE2 | remdesivir |
| Drug Name | Mechanism | Stage | (comments) | (μM) | CC₅₀ (μM) | (δ) |
| Remdesivir | RdRP inhibitor | Emergency | Cₘₐₓ ≈ 5 μM, t_{1/2} ≈ | 0.127 | >8.46 | n/a |
| (GS-5734) | | FDA | 1 h (human oral)(15) | | | |
| | | registration | | | | |
| Halofantrine | Antimalarial; | Registered | Cₘₐₓ ≈ 1μM, t_{1/2} ≈ | 1.158 | >23.927 | −3.509 |
| HCl | unknown | | 72 h (human oral)(16, 17) | | | |
| Hydroxychloroquine | Antirheumatic drug | Registered | Cₘₐₓ ≈ 1.7 μM, t_{1/2} ≈ | 0.458 | >18.345 | −1.572 |
| | | | 55 h (human oral)(18) | | | |
| Amiodarone | Antiarrhythmic | Registered | Cₘₐₓ ≈ 6.87 μM, t_{1/2} ≈ | 1.027 | 15.937 | 0.68 |
| | | | 6 h (human oral)(19) | | | |
| Nelfinavir | HIV protease | Registered | Cₘₐₓ ≈ 17 μM, t_{1/2} ≈ | >8.643 | >18.017 | −1.23 |
| mesylate | inhibitor | | 4 h (human oral)(20) | | | |
| Simeprevir | Hepatitis C NS3/4A | Registered | Cₘₐₓ ≈ 32 uM, t_{1/2} ≈ | >7.884 | >22.165 | −0.67 |
| | protease inhibitor | | 16 h (human oral)(21, 22) | | | |
| Manidipine | Ca²⁺-channel blocker | Registered | Cₘₐₓ ≈ 10 nM, t_{1/2} ≈ | >6.888 | >17.068 | −4.727 |
| | related to amlodipine | | 3 h (human, oral)(23) | | | |
| Ozanimod | S1P agonist | Registered | Cₘₐₓ ≈ 1.2 μM, t_{1/2} ≈ | 2.634 | >14.744 | −4.62 |
| | | | 21 h (human, oral)(24) | | | |
| Diclofensine | Dopamine reuptake | Phase III | Cₘₐₓ ≈ 30 nM, t_{1/2} ≈ | 6.06 | >40 | n/d |
| | inhibitor | | 15 h (human, oral)(25) | | | |
| Pancopride | 5-HT3 antagonist | Phase III | Cₘₐₓ ≈ 300 nM, t_{1/2} ≈ | >8.64 | >37.29 | 0.65 |
| | | | 16 h (human, oral)(26) | | | |
| Apilimod | PIKfyve (anti- | Phase II | Cₘₐₓ ≈ 600 nM, t_{1/2} ≈ | <0.006 | >27.804 | 3.57 |
| | inflammatory) | | 3 h (human, oral)(27) | | | |
| AQ-13 | Malaria | Phase II | Quinoline with best | 0.8 | 25.891 | −1.77 |
| | | | exposure | | | |
| LY2228820 | p38 inhibitor | Phase II | Cₘₐₓ ≈ 5 μM, t_{1/2} ≈ | 1.874 | 27.356 | −0.846 |
| | | | 145 h (human, oral)(28) | | | |
| MK-2206 | Allosteric AKT | Phase II | Cₘₐₓ ≈ 571 nM, t_{1/2} ≈ | 1.103 | >22.753 | 2.087 |
| | inhibitor | | 75 h (human, oral)(29) | | | |
| R- | AXL kinase inhibitor | Phase II | Cₘₐₓ ≈ 750 nM, t_{1/2} ≈ | 1.202 | 15.684 | 7.223 |
| 428/BGB324 | | | 80 h (oral, humans)(30) | | | |
| Hanfangchin | Ca²⁺-channel blocker | Phase I | Cₘₐₓ ≈ 0.5 μM, t_{1/2} ≈ | 1.177 | >18.921 | −3.61 |
| A | | | 20 h (rats, oral)(31) | | | |
| L 796568 | Beta-3 adrenergic | Phase I | Cₘₐₓ ≈ 0.3 μM, t_{1/2} ≈ | 1.045 | 5.512 | n/d |
| | receptor agonist | | 14 h (dog, oral)(32) | | | |
| NCO 700 | Cathepsin B inhibitor | Phase I | Unknown | 0.79 | >39.82 | 0.209 |
| Osimertinib | EGFR (Thr790Met | Approved | Cₘₐₓ ≈ 1.5 μM, t_{1/2} ≈ | 0.962 | >18.07 | −0.722 |
| | Mutant) Inhibitor | | 42 h (human, oral)(33, 34) | | | |
| SMN-C3 | SMA splicing | Phase I | Generally high for | 1.399 | 7.16 | −0.37 |
| | modifier | | class; Cₘₐₓ ≈ 2 μM, | | | |
| | | | t_{1/2} ≈ (mouse, oral)(35) | | | |
| GS-9901 | Phosphatidylinositol | Phase I | Cₘₐₓ ≈ 1.8 μM, t_{1/2} ≈ | 3.807 | >31.113 | n/d |
| | 3-kinase delta | | 2 h (rat, oral)(36) | | | |
| | inhibitor | | | | | |
| GW-803430 | Melanin- | Phase I | Cₘₐₓ ≈ 585 nM, (rat, | 1.696 | 18.919 | n/d |
| | Concentrating | | oral)(37) t_{1/2} ≈ | | | |
| | Hormone MCH-R1 | | 11 h (mouse)(38) | | | |
| | (SLC-1) Receptor | | | | | |
| | Antagonist | | | | | |
| SAX-187 | 5 Hydroxytryptamine | Phase I | Cₘₐₓ = 0.4 μM, t_{1/2} ≈ | 1.846 | 7.428 | n/d |
| | 6 receptor agonist | | 3 h (rat, oral)(39) | | | |
| YM-161514 | Ca-channel blocker | Phase I | Unknown; Generally | 1.863 | 11.334 | 0.60 |
| | | | low for class | | | |
| Dutacatib | Cathepsin K inhibitor | Discovery | Unknown | 2.689 | >36.111 | 2.955 |
| KC 11404 | Lipoxygenase 5 | Discovery | Unknown | 2.496 | >20.94 | −2.817 |
| | inhibitor | | | | | |
| NNC | Sodium and calcium | Discovery | Unknown | 2.747 | >15.351 | n/d |
| 090026 | channel inhibitor | | | | | |

Cₘₐₓ, maximum serum concentration;
t_{1/2}, time to half Cₘₐₓ;
tₘₐₓ, time to Cₘₐₓ;
n/a, not applicable;
n/d, not determined

Because viruses rely on host machinery for replication, it was not unexpected that many of the compounds with antiviral activity also affected vital host processes. Interestingly, this toxicity was sometimes masked in infected cells, as reduction of viral infection by compounds like the protein synthesis inhibitor puromycin and even hydroxychloroquine provided a benefit to cell health in the context of infection but not in uninfected cells (FIG. 2, panel C).

Between the small pilot and the ReFRAME screen, we identified 76 (75 unique as two different lots of GW-803430 were identified) potent (EC₅₀<9.6 μM) and selective (CC₅₀/EC₅₀>10 or CC₅₀>39.8 μM) compounds, and 135 compounds that were either only weakly active (EC₅₀>9.6 μM) or potent, but not adequately selective (EC₅₀<9.6 μM, CC₅₀/EC₅₀<10) (FIG. 2, panels B and D, Table 1). The top four classes of potent and selective compounds were oncolytic compounds, ion channel modulators, anti-psychotics and receptor binding compounds (FIG. 2, panel D). For weakly active or nonselective hits, the top four categories likewise included oncolytic compounds, ion channel modulators, anti-psychotics as well as signal transduction modulators. A fifth of potent and selective hits could be classified as oncolytic drugs, further reflecting the reliance of the virus on host cell processes present in rapidly proliferating cells. The identification of compounds belonging to anti-psychotic, cardiovascular, and even anti-parasitic (neglected tropical diseases) classes can reflect the cationic amphiphilic nature of some of these molecules and their ability to accumulate in and impact acidic intracellular compartments (e.g. late endosomes/lysosomes). Resultant dysregulation of the endo-lysosomal pathway and lipid homeostasis has been suggested to impair viral entry and/or replication (8) and this mode of action is contemplated for amiodarone and hydroxychloroquine, both identified here as potent and selective hits against SARS-CoV-2 in our screen (Tables 1 and 3). We also identified two selective estrogen receptor modulators (bazedoxifene, EC₅₀=3.47 μM and raloxifene EC₅₀=4.13 μM), a class of compounds previously found to inhibit Ebola virus infection (9).

Additional embodiments for use in any of the methods or compositions described herein include further reconfirmed hits from the ReFRAME screen. These are presented in Table 4 with corresponding data from the SARS-CoV-2/HeLa-ACE2 high-content screening assay described herein and a SARS-CoV-2/Calu-3 high-content screening assay (see Examples).

TABLE 4
Reconfirmed hits from a screen of the ReFRAME library in Calu-3 cells, anti-viral activities

| | | | | | HeLa-ACE2 | | |
|---|---|---|---|---|---|---|---|
| | | Calu-3 (aggregated | | | (aggregated data, | | |
| Compound | | data, Geomean, n = 3) | | | Geomean, n = 3) | | |
| | | | | | | | HeLa- |
| | | | | Calu3 | | Un- | ACE2 |
| | | | | SI | | infected | SI |
| | | CoV-2 | Uninfected | CC₅₀/ | CoV-2 | Tox | CC₅₀/ |
| | | EC₅₀ | Tox CC₅₀ | CoV-2 | EC₅₀ | CC₅₀ | CoV-2 |
| Name | Structure | [μM] | [μM] | EC₅₀ | [μM] | [μM] | EC₅₀ |
| RWJ- 56423 | 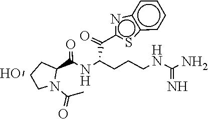 | 1.228 | >29.90 | >24 | >9.59 | >39.8 | NA |
| TO-195 | 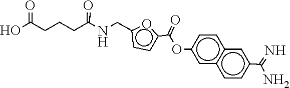 | 0.657 | >29.90 | >45 | >9.59 | >39.8 | >6 |
| Avoralstat | 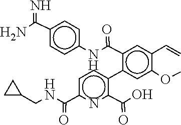 | 0.387 | >29.90 | >77 | >9.59 | >39.8 | NA |
| YM 60828 | 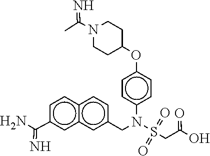 | 3.377 | >29.90 | >9 | >9.59 | >39.8 | NA |
| UK- 356202 | 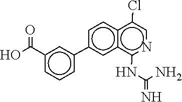 | 1.916 | >29.90 | >15 | >9.59 | >39.8 | NA |
| Bardoxolone Methyl | 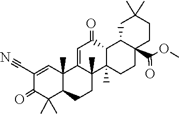 | 0.079 | 3.055 | 38.47 | 0.541 | 3.098 | 5.73 |
| MK-8722 | 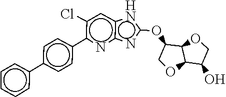 | 2.733 | >29.90 | >10 | >9.59 | 37.433 | NA |
| AZ- 11713908 | 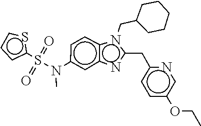 | 3.535 | >29.90 | >8 | >9.59 | 32.963 | NA |
| cerivastatin (sodium salt) | 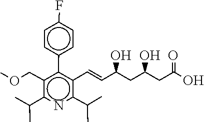 | 0.348 | >29.90 | >85 | >9.59 | 0.308 | NA |
| CYMARINE | 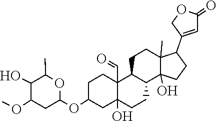 | 0.135 | 5.868 | 43.53 | 0.237 | 0.287 | 1.21 |
| Ono- 3307 | 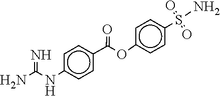 | 1.602 | >29.90 | >16 | >9.59 | >39.8 | NA |
| AMA- 0076 | 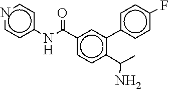 | 3.868 | >29.90 | >16 | >9.59 | 35.846 | NA |
| Mitoguazone | 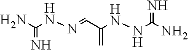 | 0.446 | >29.90 | >67 | >9.59 | >39.8 | NA |
| Resiniferatoxin | 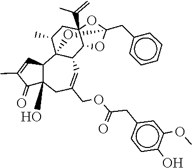 | 0.903 | 17.279 | 19.13 | 1.296 | 7.171 | 5.53 |
| DEAZANEPLANOCINA | 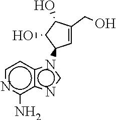 | 0.057 | 10.346 | 182.24 | >9.59 | 7.951 | NA |
| OLIGOMYCINA | 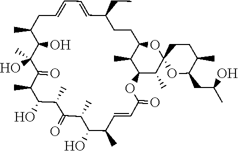 [SMILES: CC[C@@H]1/C=C/C=C/C[C@H](C)[C@@H](O)[C@](C)(O)C(=O)[C@H](C)[C@@H](O)[C@H](C)C(=O)[C@H](C)[C@@H](O)[C@H](C)/C=C/C(=O)O[C@H]2[C@@H](C)[C@@H](CC1)O[C@@]1(CC[C@@H](C)[C@@H](C[C@H](C)O)O1)[C@@H]2C] | 0.007 | >29.90 | >4000 | >9.59 | 36.040 | NA |
| Oligomycin B | 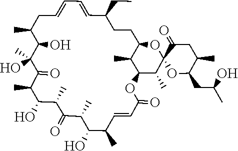 [SMILES: CC[C@@H]1/C=C/C=C/C[C@H](C)[C@@H](O)[C@](C)(O)C(=O)[C@H](C)[C@@H](O)[C@H](C)C(=O)[C@H](C)[C@@H](O)[C@H](C)/C=C/C(=O)O[C@H]2[C@@H](C)[C@@H](CC1)O[C@]1(O[C@H](C[C@H](C)O)[C@H](C)CC1=O)[C@@H]2C] | 0.005 | 24.477 | 4539.49 | >9.59 | 21.980 | NA |
| BMS- 223131 | 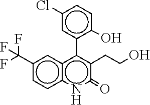 | 5.143 | >29.90 | >5.8 | >9.59 | 21.425 | NA |
| Lestaurtinib | 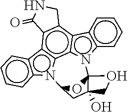 | 0.674 | 20.123 | 29.86 | 0.546 | 0.758 | 1.39 |
| Gemcitabine elaidate | 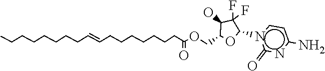 | 0.324 | >29.90 | >92 | >9.59 | 6.006 | NA |
| Bruceantin | 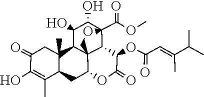 [SMILES: COC(=O)[C@@]12OC[C@]34[C@H]([C@@H](O)[C@@H]1O)[C@@]1(C)CC(=O)C(O)=C(C)[C@@H]1C[C@H]3OC(=O)[C@H](OC(=O)/C=C(\C)C(C)C)[C@@H]24] | 0.019 | 0.973 | 50.22 | 0.006 | 0.117 | 20.18 |
| LANATOSIDE | 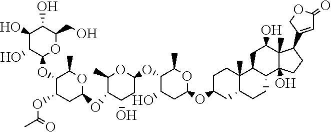 [SMILES: CC(=O)O[C@H]1C[C@H](O[C@H]2[C@@H](O)C[C@H](O[C@H]3[C@@H](O)C[C@H](O[C@H]4CC[C@@]5(C)[C@H](CC[C@@H]6[C@@H]5C[C@@H](O)[C@]5(C)[C@@H](C7=CC(=O)OC7)CC[C@]65O)C4)O[C@@H]3C)O[C@@H]2C)O[C@H](C)[C@H]1O[C@@H]1O[C@H](CO)[C@@H](O)[C@H](O)[C@H]1O] | 0.194 | 6.206 | 31.97 | 0.206 | 1.351 | 6.56 |
| ANISOMYCIN | 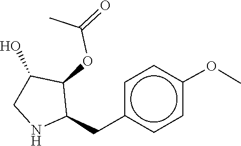 | 0.127 | 5.983 | 46.98 | 0.066 | 0.189 | 2.85 |
| Cephaeline | 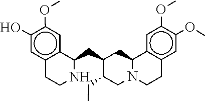 | 0.142 | 4.963 | 34.84 | 0.017 | 0.212 | 12.66 |
| SR-26050 | 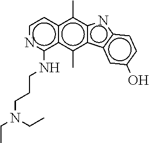 | 0.503 | 5.837 | 11.61 | 0.359 | 0.602 | 1.68 |
| Emetine | 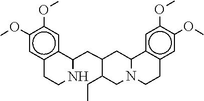 | 0.444 | 8.871 | 19.97 | 0.060 | 0.340 | 5.66 |
| Phorbol 12- myristate 13- acetate | 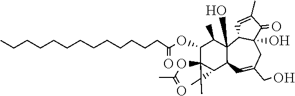 [SMILES: CCCCCCCCCCCCCC(=O)O[C@@H]1[C@@H](C)[C@@]2(O)[C@@H](C=C(CO)C[C@]3(O)C(=O)C(C)=C[C@@H]23)[C@@H]2C(C)(C)[C@]12OC(C)=O] | 0.016 | 22.452 | 1384.37 | 0.070 | 0.201 | 2.89 |
| ARN-810 | 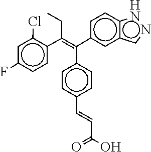 | 1.104 | 23.843 | 21.60 | >9.59 | 10.951 | NA |
| VX-803 | 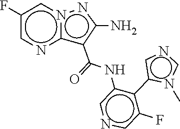 | 0.661 | >29.90 | >45 | >9.59 | 13.438 | NA |
| E-7090 | 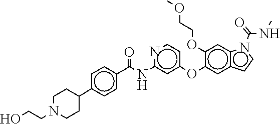 | 0.753 | 14.980 | 19.88 | 2.645 | 21.190 | 8.01 |
| Verosudil | 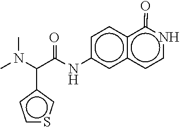 | 3.507 | 18.887 | 5.39 | >9.59 | >39.8 | NA |
| Cycloheximide | 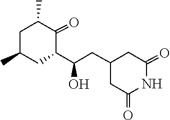 [SMILES: C[C@@H]1C[C@@H]([C@H](O)CC2CC(=O)NC(=O)C2)C(=O)[C@@H](C)C1] | 0.195 | 2.435 | 12.50 | 0.634 | 1.469 | 2.32 |
| PYRIDABEN | 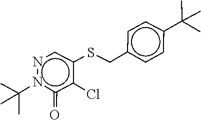 | 0.205 | 8.993 | 43.89 | >9.59 | >39.8 | NA |
| Antimycin A | 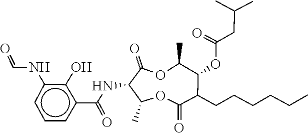 | 0.028 | 0.608 | 22.00 | >9.59 | >39.8 | NA |
| Antimycin A3 | 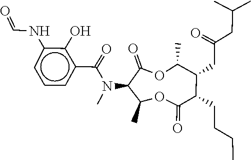 | 0.036 | 3.897 | 109.64 | >9.59 | >39.8 | NA |
| AGN-194310 | 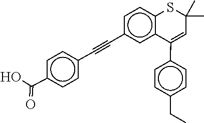 | 0.597 | >29.90 | >50 | >9.59 | >39.8 | NA |
| TUBERCIDIN | 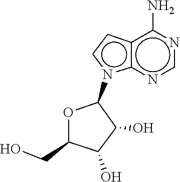 | 0.111 | 1.072 | 9.68 | 0.183 | 0.557 | 3.04 |
| ON-09310 | 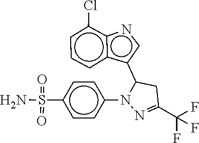 | 1.808 | 16.886 | 9.34 | 3.721 | 8.197 | 2.20 |
| tanaproget | 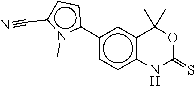 | 2.439 | 19.278 | 7.99 | 0.141 | 0.128 | 0.91 |
| auranofin | 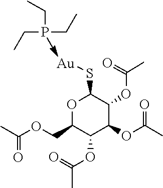 | 0.525 | 3.129 | 7.87 | 0.988 | 0.634 | 0.64 |
| Analog of OGT-719 | 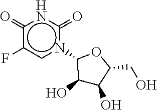 | 1.173 | 8.726 | 7.44 | 4.017 | 0.109 | 0.03 |
| Azathioprin | 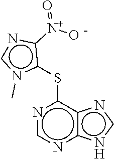 | 0.482 | 3.546 | 7.35 | 5.083 | 38.970 | 7.67 |
| Mercaptopurine | 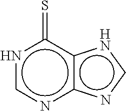 | 0.197 | 1.368 | 6.95 | 3.567 | 19.639 | 5.51 |
| FO 152 | 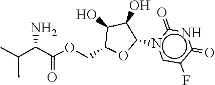 | 2.500 | 17.223 | 6.89 | 8.931 | 0.533 | 0.06 |
| Cloturin | 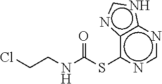 | 0.304 | 2.012 | 6.62 | 3.880 | 31.546 | 8.13 |
| Tioguanine | 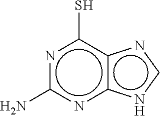 | 0.127 | 0.822 | 6.45 | 0.787 | 2.820 | 3.58 |
| Metildigoxin | 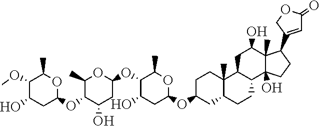 [SMILES: CO[C@H]1[C@@H](O)C[C@H](O[C@H]2[C@@H](O)C[C@H](O[C@H]3[C@@H](O)C[C@H](O[C@H]4CC[C@@]5(C)[C@H](CC[C@@H]6[C@@H]5C[C@@H](O)[C@]5(C)[C@@H](C7=CC(=O)OC7)CC[C@]65O)C4)O[C@@H]3C)O[C@@H]2C)O[C@@H]1C] | 0.399 | 1.239 | 6.22 | 0.284 | 0.159 | 0.56 |
| 6- Methyl mercaptopurineriboside (metabolite) | 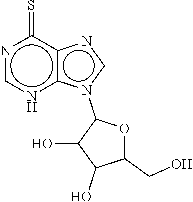 | 0.207 | 1.200 | 5.80 | 3.599 | 15.375 | 4.27 |
| BN- 82685 | 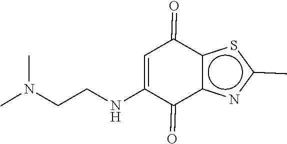 | 3.048 | 5.964 | 5.69 | 1.534 | 4.655 | 3.04 |
| Peruvoside (analog) | 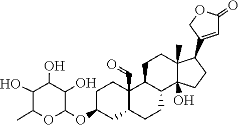 [SMILES: CC1OC(O[C@H]2CC[C@@]3(C=O)[C@H](CC[C@@H]4[C@@H]3CC[C@]3(C)[C@@H](C5=CC(=O)OC5)CC[C@]43O)C2)C(O)C(O)C1O] | 0.251 | 1.407 | 5.61 | 0.246 | 0.345 | 1.40 |
| ethinyl estradiol sulfonate | 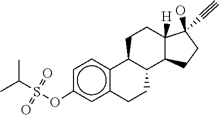 | 1.227 | 6.855 | 5.59 | >9.59 | 20.449 | NA |
| Mavatrep | 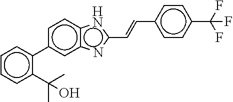 | 2.659 | 14.621 | 5.50 | >9.59 | 22.498 | NA |
| ZD 2138 | 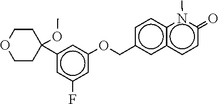 | 2.787 | 14.424 | 5.18 | >9.59 | 28.748 | NA |
| TULOPAFANT | 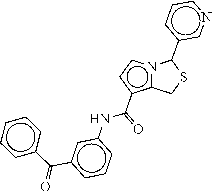 | 2.917 | 14.621 | 5.01 | >9.59 | 36.578 | NA |
| oxametacin | 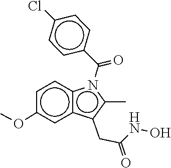 | 3.565 | 17.746 | 4.98 | 9.139 | 5.874 | 0.64 |
| LANATOSIDE B | 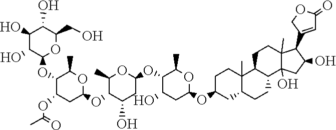 | 0.688 | 3.370 | 4.90 | 0.803 | 1.747 | 2.175 |
| Digoxin | 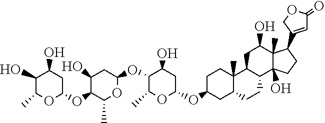 [SMILES: C[C@H]1O[C@@H](O[C@H]2[C@@H](O)C[C@@H](O[C@@H]3[C@@H](O)C[C@H](O[C@H]4CC[C@@]5(C)[C@H](CC[C@@H]6[C@@H]5C[C@@H](O)[C@]5(C)[C@@H](C7=CC(=O)OC7)CC[C@]65O)C4)O[C@@H]3C)O[C@@H]2C)C[C@H](O)[C@@H]1O] | 0.17 | >5.035 | >29.80 | 0.166 | 0.12 | 0.72 |
| CFI- 400945 | 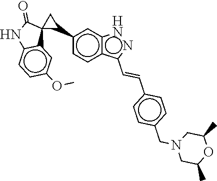 | 3.36 | >19.756 | >5.88 | >7.001 | 7.176 | <1.02 |
| INS- 117548 | 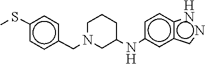 | >3.546 | >29.875 | >8.42 | >9.59 | >39.8 | 4.15 |
| SAR- 407899 | 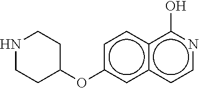 | >3.845 | >29.867 | >7.77 | >9.59 | >38.66 | 4.03 |
| GENZ- 29155 | 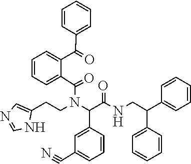 [SMILES: N#Cc1cccc(C(C(=O)NCC(c2ccccc2)c2ccccc2)N(CCc2cnc[nH]2)C(=O)c2ccccc2C(=O)c2ccccc2)c1] | >4.659 | >29.867 | >6.41 | >9.59 | >38.47 | 4.01 |
| Elubrixitosylate | 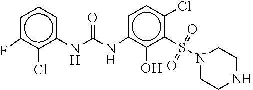 | 6.908 | >29.564 | >4.28 | >9.59 | >39.8 | 4.35 |

Among the identified hits, according to various embodiments are newly identified and approved oral drugs halofantrine HCl, amiodarone, nelfinavir mesylate, simperevir, manidipine, and ozanimod, due to their relatively high exposures or a long history of use as therapeutic agents and therefore potential to be quickly repurposed as COVID-19 treatments following further efficacy vetting in animal models. For example, the viral protease inhibitors nelfinavir and simeprevir exhibit excellent exposures.

In another embodiment, the compound is the selective sphingosine-1-phosphate (S1P1) receptor modulator ozanimod. Selective S1P1 agonists have been shown to provide significant protection against influenza virus infection in murine models by reducing inflammation at the site of infection (reducing release of cytokines by pulmonary endothelial cells and infiltration of lymphocytes into the lungs) (10), and thus ozanimod can serve as an excellent combination partner for a direct-acting antiviral drug.

In accordance with another embodiment, the compound administered in the methods described herein is the approved drug amiodarone, which has excellent exposure (Cₘₐₓ˜684 μM), or the approved calcium-channel blocker manidipine, which has low exposure but can improve COVID-19 disease outcomes for patients with hypertension. Amiodarone is further identified as having broad-spectrum antiviral activity in an in vitro screen (11).

Nineteen other compounds in various stages of development such as apilimod (assay control that may inhibit viral entry through disruption of endo-lysosomal trafficking, as found for filoviruses (12)), the protease inhibitors NCO 700 (cathepsin B) and dutacatib (cathepsin K), which can also impact viral entry, all can show efficacy due to their potency or pharmacokinetic profiles (Table 3). Most of these, except for the very potent apilimod, had modest EC₅₀s>1 μM that did not surpass the potency of remdesivir.

The present disclosure also provides in some embodiments a method for reducing the likelihood of a pathogenic infection from occurring in a subject or reducing transmission of a pathogen from an infected subject to other subjects. The method comprises administering to the subject at least one compound listed in Table 1 or Table 4, optionally in combination with at least one anti-infective agent as described herein.

### Combination Therapy

In various embodiments, the methods of the present disclosure further comprise administering an anti-infective agent. The anti-infective agent can be administered concomitantly with at least one compound as described herein (Table 1 and Table 4), such as in the same formulation or dosage form. Alternatively, the anti-infective agent can be administered before or after the compound.

In some embodiments, the anti-infective agent is selected from the group consisting of entry-inhibiting drugs (including enfuvirtide), uncoating inhibiting drugs (including amantadine, rimantadine, and pleconaril), reverse transcriptase inhibiting drugs (including acyclovir, zidovudine, and lamivudine), antisense drugs (including fomivirsen), ribozyme drugs, protease inhibitors, assembly inhibiting drugs (including rifampicin), and release inhibiting drugs.

In some embodiments, an additional agent is chosen from dexamethasone, amodiaquine,

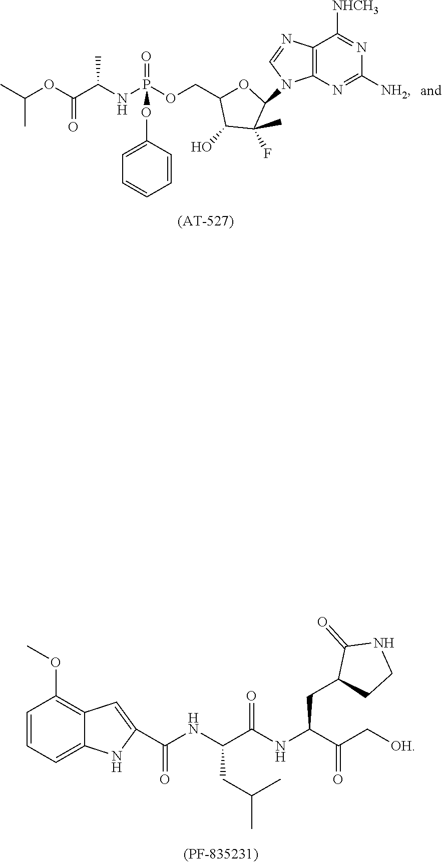 [SMILES: CNc1nc(N)nc2c1ncn2[C@@H]1OC(CO[P@@](=O)(N[C@@H](C)C(=O)OC(C)C)Oc2ccccc2)[C@@H](O)[C@@]1(C)F ; COc1cccc2[nH]c(C(=O)N[C@@H](CC(C)C)C(=O)N[C@@H](C[C@@H]3CCNC3=O)C(=O)CO)cc12]

In some embodiments, the additional anti-infective agent is an anti-viral agent. In some embodiments, the anti-viral agent is selected from the group consisting of Abacavir, Acyclovir (Aciclovir), Adefovir, Amantadine, Ampligen, Amprenavir (Agenerase), Arbidol, Atazanavir, Atripla, Balavir, Baloxavir marboxil (Xofluza), Biktarvy, Boceprevir (Victrelis), Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Favipiravir (T-705; 6-fluoro-3-hydroxy-2-pyrazinecarboxamide), Fomivirsen, Fosamprenavir, Foscarnet, Fosfonet, Fusion inhibitor, Ganciclovir (Cytovene), Ibacitabine. Ibalizumab (Trogarzo), Idoxuridine, Imiquimod, Imunovir, Indinavir, Inosine, Integrase inhibitor, Interferon type I, Interferon type II, Interferon type III, Interferon, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues. Oseltamivir (Tamiflu), Peginterferon alfa-2a, Peginterferon alfa-2b. Penciclovir, Peramivir (Rapivab), Pleconaril, Podophyllotoxin, Protease inhibitor (pharmacology), Pyramidine, Raltegravir, Remdesivir, Reverse transcriptase inhibitor, Ribavirin, Rilpivirine (Edurant), Rimantadine, Ritonavir, Saquinavir, Simeprevir (Olysio), Sofosbuvir, Stavudine, Synergistic enhancer (antiretroviral), Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine.

The requirement for intravenous administration and potentially limited efficacy of remdesivir prompted further investigation into alternative or supplemental therapies. Thus, in accordance with various embodiments, the present disclosure provides compounds as disclosed herein as partners with remdesivir in a combination therapy.

A combination therapy as disclosed herein can increase efficacy of treatment while reducing drug dose of either or both combination partners, and thus prevent side effects that may be associated with administration of higher doses. Drug combinations can also slow the acquisition of drug resistance. Drug synergy, which is defined as the increase in activity of the combination therapy beyond what is expected of an additive interaction is rare, and yet additive effects themselves can improve therapy regimens. Conversely, antagonism, the inhibition of activity of the overall combination beyond what would be expected if the compounds acted independently, is an undesirable property.

Thus, to identify synergistic, additive, and antagonistic interactions between the FDA-approved remdesivir and ReFRAME hits, we performed synergy interactions studies in a checkerboard experiment, comparing full dose response of remdesivir against the dose responses of 11 hits with attractive safety and pharmacokinetic profiles in a 10×10 matrix (FIG. 2, panel E). We used the synergfinder package (13) in R to assess the interactions between the tested compounds using the Zero Interaction Potency Model (ZIP) (14), where a δ score>10 indicates likely synergy, δ<−10 indicates antagonism, and 6 between −10 and 10 suggests an additive interaction. The results showed that several exemplary combinations are additive (FIG. 2, panels E and F, Table 1).

This screen also identified the nucleoside analog riboprine (N6-isopentenyladenosine, previously investigated as an antineoplastic agent, for treatment of nausea and surgical site infections, and a component of CitraNatal 90 DHA, a prescription prenatal/postnatal multivitamin/mineral tablet) and a folate antagonist 10-deazaaminopterin (an antineoplastic compound currently in Phase II stage of development) as having activities that synergized with those of remdesivir. The synergistic effects for both compounds were observed across specific concentrations, signified as peaks within a 3-dimensional synergy score landscape.

Riboprine achieved maximal (100%) efficacy over the range of concentrations tested, but addition of EC2 of remdesivir shifted its EC50 from 12 μM to 3.6 μM, and addition of EC24 of remdesivir increased its potency further to EC50=1.6 μM. 10-deazaaminopterin showed only 40% maximal efficacy over the range of concentrations tested, but the addition of EC2 of remdesivir caused an increase of maximal efficacy from 40% to nearly 65% (where a shift of 2% would be expected) and addition of EC24 of remdesivir increased maximal efficacy of the combination from 40% to >80%.

### Pharmaceutical Composition

The present disclosure provides in various embodiments a pharmaceutical composition comprising a therapeutically effective amount of at least one compound selected from Table 1 or Table 4 as described herein, a therapeutically effective amount of an anti-infective agent as described herein, and a pharmaceutically acceptable carrier. In some embodiments, the composition further contains, in accordance with accepted practices of pharmaceutical compounding, one or more additional pharmaceutically acceptable excipients, diluents, adjuvants, stabilizers, emulsifiers, preservatives, colorants, buffers, and flavor imparting agents.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound that is administered, including all active ingredients of a combination therapy, is governed by such considerations, and is the minimum amount necessary to elicit an anti-infective, e.g., anti-viral, effect. Such amount may be below the amount that is toxic to normal cells, or the subject as a whole. Generally, the initial therapeutically effective amount of a compound of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 1 mg to about 1000 mg of a compound of the present disclosure. In another embodiment, such dosage forms contain from about 50 mg to about 500 mg of a compound of the present disclosure. In yet another embodiment, such dosage forms contain from about 25 mg to about 200 mg of a compound of the present disclosure. In still another embodiment, such dosage forms contain from about 10 mg to about 100 mg of a compound of the present disclosure. In a further embodiment such dosage forms contain from about 5 mg to about 50 mg of a compound of the present disclosure.

The compositions can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions in accordance with the present disclosure include without limitation tablets, troches, lozenges, aqueous or oily suspensions, dispersible powders or granules, emulsion, hard or soft capsules, syrups or elixirs.

Encompassed within the scope of the present disclosure are pharmaceutical compositions suitable for single unit dosages that comprise a compound of the disclosure or its pharmaceutically acceptable stereoisomer, salt, or tautomer and a pharmaceutically acceptable carrier.

Compositions suitable for oral use may be prepared according to any method known to the art for the manufacture of pharmaceutical compositions. For instance, liquid formulations of the compounds contain one or more agents selected from the group consisting of sweetening agents, flavoring agents, coloring agents and preserving agents in order to provide pharmaceutically elegant and palatable preparations of the arginase inhibitor.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. Tc oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensaturatedion products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the present disclosure may also be administered in the form of suppositories for rectal administration of the compounds. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### EXAMPLES

The following examples are illustrative and non-limiting to the scope of the compositions, methods, and formulations described herein.

Virus generation. Vero E6 cells (ATCC CRL-1586) were plated in a T225 flask with complete DMEM (Corning 15-013-CV) containing 10% FBS, 1×PenStrep (Corning 20-002-CL), 2 mM L-Glutamine (Corning 25-005-CL) overnight at 37° C. 5% CO₂. The media in the flask was removed and 2 mL of SARS-CoV-2 strain USA-WA 1/2020 (BEI Resources NR-52281) in complete DMEM was added to the flask at an MOI of 0.5 and was allowed to incubate for 30 minutes at 34° C. 5% CO₂. After incubation, 30 mL of complete DMEM was added to the flask. The flask was then placed in a 34° C. incubator at 5% CO₂ for 5 days. On day 5 post infection the supernatant was harvested and centrifuged at 1,000×g for 5 minutes. The supernatant was filtered through a 0.22 μM filter and stored at −80° C.

The ReFRAME library: Compound management, drug annotation and screen data access. The ReFRAME library collection consists of nearly 12,000 high-purity compounds (>95%) dissolved in high-quality dimethyl sulfoxide (DMSO). Compound quality control was performed by liquid chromatography-mass spectrometry and/or ¹H-NMR when required. The library was prepared at two concentrations, 2 and 10 mM, to support low-concentration (2-10 μM) and high-concentration (10-50 μM) screening formats. Echo-qualified 384-well low dead volume plus microplates (LP-0200-BC; Labcyte Inc.) were used as the library source plates to support acoustic transfer with an Echo 555 Liquid Handler (Labcyte Inc.). Compounds not soluble in DMSO were plated in water (129 compounds); compounds lacking long-term solubility in DMSO were suspended just before dispensing to avoid precipitation (71 compounds).

Associated compound annotations (Table 1) are supported by three widely used commercial drug competitive intelligence databases: Clarivate Integrity. GVK Excelra GoStar, and Citeline Pharmaprojects. As available, annotation data may include status of clinical development and highest stage of development achieved, mechanism of action, drug indication(s), and route of administration.

HeLa-ACE2 stable cell line. HeLa-ACE2 cells were generated through transduction of human ACE2 lentivirus. The lentivirus was created by co-transfection of HEK293T cells with pBOB-hACE2 construct and lentiviral packaging plasmids pMDL, pREV, and pVSV-G (Addgene) using Lipofectamine 2000 (Thermo Fisher Scientific, 11668019). Supernatant was collected 48 h after transfection then used to transduce pre-seeded HeLa cells. 12 h after transduction stable cell lines were collected, scaled up and stored. Cells were maintained in DMEM (Gibco, 11965-092) with 10% FBS (Gibco, 10438026) and 1×sodium pyruvate (Gibco, 11360070) at 37° C. 5% CO2.

SARS-CoV-2/HeLa-ACE2 high-content screening assay. Compounds were acoustically transferred into 384-well μclear-bottom plates (Greiner, Part. No. 781090-2B). HeLa-ACE2 cells were seeded in 13 μL DMEM with 2% FBS at a density of 1.0×103 cells per well. Plated cells were transported to the BSL3 facility where 13 μL of SARS-CoV-2 diluted in assay media was added per well at an assay multiplicity of infection (MOI)=2.2 for primary screening, adjusted to 0.65 for powder reconfirmation. Plates were incubated for 24 h at 34° C. 5% CO2, and then fixed with final concentration of 4% formaldehyde for 1 h at 34° C. 5% CO2. Plates were washed with 1×PBS 0.05% Tween 20 in between fixation and subsequent primary and secondary antibody staining. Human polyclonal plasma diluted 1:500 in Perm/Wash buffer (BD Biosciences 554723) was added to the plate and incubated at RT for 2 h. Eight μg/mL (1:250 dilution) of goat anti-human H+L conjugated Alexa 488 (Thermo Fisher Scientific A 11013) together with 8 μM of antifade-46-diamidino-2-phenylindole (DAPI; Thermo Fisher Scientific D1306) in SuperBlock T20 (PBS) buffer (Thermo Fisher Scientific 37515) was added to the plate and incubated at RT for 1 h in the dark. Plates were imaged using the ImageXpress Micro Confocal High-Content Imaging System (Molecular Devices) with a 10× objective, with 4 fields imaged per well. Images were analyzed using the Multi-Wavelength Cell Scoring Application Module (MetaXpress), with DAPI staining identifying the host-cell nuclei (the total number of cells in the images) and the SARS-CoV-2 immunofluorescence signal leading to identification of infected cells.

Time of addition (TOA) assay. HeLa-ACE2 cells were infected with SARS-CoV-2 in suspension in assay medium (DMEM with 2% FBS) at an MOI of 1.5 for 1 h at 34° C. 5% CO2, then extensively washed with PBS and plated in assay-ready 384-well plates pre-spotted with compounds as for the standard HeLa-ACE2 infection assay. For the time course experiment, cells were fixed with a final concentration of 4% formaldehyde at 4, 5, 6, 7, 8, 10, 11, 12, and 24 hpi and stained and imaged as for the standard infection assay to determine optimal timepoint for TOA assay. TOA assay was performed in the same manner, with cells fixed at 10 hpi.

Calu-3 high-content screening assay. The assay is carried out as outlined for the HeLa-ACE2 assay, with the following exceptions. Calu-3 cells (ATCC HTB-55), a kind gift from Dr. Catherine Chen at NCATS/NIH and Dr. Juan Carlos de la Torre at Scripps Research, were seeded at a density of 5,000 cells per 20 μL per well in assay media (MEM with 2% FBS) and SARS-CoV-2 diluted in assay media was added at an MOI between 0.75 and 1 to achieve ˜30-60% infected cells. Plates were incubated for 48 h at 34° C. 5% CO2, and then fixed with a final concentration of 4% formaldehyde. Fixed cells were stained and imaged as in the HeLa-ACE2 assay.

Uninfected host cell cytotoxicity counter screens. For HeLa-ACE2 cells, compounds were acoustically transferred into 1,536-well μclear plates (Greiner Part. No. 789091). HeLa-ACE2 cells were maintained as described for the infection assay and seeded in the assay-ready plates at 400 cells/well in DMEM with 2% FBS and plates were incubated for 24 h at 37° C. 5% CO2. To assess cell viability, the Image-iT DEAD green reagent (Thermo Fisher) was used according to manufacturer instructions. Cells were fixed with 4% paraformaldehyde, and counterstained with DAPI. Fixed cells were imaged using the ImageXpress Micro Confocal High-Content Imaging System (Molecular Devices) with a 10×objective, and total live cells per well quantified in the acquired images using the Live Dead Application Module (MetaXpress).

For Calu-3 cells, compounds were acoustically transferred into 1,536-well plates (Corning No. 9006BC) before seeding Calu-3 cells in assay media (MEM with 2% FBS) at a density of 600 cells per 5 μL per well. Plates were incubated for 48 h at 37° C. 5% CO₂. To assess cell viability, 2 μL of 50% Cell-Titer Glo (Promega No G7573) diluted in water was added to the cells and luminescence measured on an EnVision Plate Reader (Perkin Elmer).

HepG2 (ATCC HB-8065) and HEK293T (ATCC CRL-3216) mammalian cell lines were maintained in Dulbecco's Modified Eagle Medium (DMEM, Gibco) with 10% heat-inactivated HyClone FBS (GE Healthcare Life Sciences), 100 IU penicillin, and 100 mg/mL streptomycin (Gibco) at 37° C. with 5% CO2 in a humidified tissue culture incubator. To assay mammalian toxicity of hit compounds, 750 HepG2 and 375 HEK293T cells/well were seeded, respectively, in assay media (DMEM, 2% FBS, 100 IU penicillin, and 100 mg/mL streptomycin) in 1536-well, white, tissue culture-treated, solid bottom plates (Corning, 9006BC) that contained acoustically transferred compounds in a three-fold serial dilution starting at 40 μM. After a 72-h incubation, CellTiter-Glo Luminescent Cell Viability Assay (Promega No G7573) was used to quantify cell viability as for Calu-3 cells.

SARS-CoV-2 primary ALI HBEC model. Normal primary human bronchial epithelial cells (HBECs) (Lonza) were cultured in Millicell-96 cell culture insert plates with 1 μm PET filters (Sigma) at an air liquid interface for at least 4 weeks using PneumaCult™-ALI Medium (Stemcell Technologies). Briefly, the HBECs were first expanded in cell culture flasks before seeding 10,000 cells per well submerged in PneumaCult™-Ex Plus Medium. After 1 week, the cells were switched into PneumaCult™-ALI Medium and medium was removed from the apical surface. The air liquid interface was maintained, and the medium exchanged every 2-3 days for at least 4 weeks to allow for differentiation of the cells. Prior to infection, the apical surface was rinsed once with DPBS and compounds were added to the basolateral chamber. 20,000 PFU SARS-CoV-2 strain USA-WA1/2020 were added to the apical surface in 50 μL PBS and allowed to incubate for 2 h. The inoculum was then removed, and the cells rinsed once with DPBS. The medium was exchanged, and fresh compound added at 24 and 48 h post-infection. Apical washes were collected at 72 h post-infection by adding 100 μL DPBS to the apical surface for 15 minutes. RNA was isolated from the apical washes using the PureLink™ Pro 96 Viral RNA/DNA Purification Kit (Thermo Fisher) and analyzed for viral RNA levels by RT-qPCR using the SuperScript™ III Platinum™ One-Step qRT-PCR Kit (Thermo Fisher) and the 2019-nCoV N1 CDC Primers and Probe set (Integrated DNA Technologies). A standard curve was generated by isolating RNA from serial dilutions of the stock virus and used to determine the PFU equivalents/mL for each sample. The viral load reductions were then determined for each experimental compound treatment compared to the neutral DMSO control and plotted in log scale. Cytotoxicity was assessed by measuring LDH activity in the basolateral media using a Cytotoxicity Detection kit (LDH) (Sigma) following the manufacturer's instructions. Averages were taken for the experimental samples and presented as a percentage of the positive control puromycin. Technical triplicates were run for both antiviral and cytotoxicity readouts.

Golden Syrian Hamster SARS-CoV-2 efficacy model. Eight-week old Golden Syrian hamsters (Charles River) (five per group) were dosed per os (PO) as indicated. Four hours post first dose, hamsters were infected through intranasal installation of 106 total PFU per animal in 100 μL of DMEM. Hamsters were dosed with compound bidaily (BID) and weighed for the duration of the study. At day 5 post-infection, the masters were sacrificed, and lung tissue was isolated for analysis. The research protocol was approved and performed in accordance with Scripps Research TACUC Protocol #20-0003.

Lung Viral Titer Determination. SARS-CoV2 titers were measured by homogenizing organs in DMEM 2% FCS using 100 μm cell strainers (Myriad 2825-8367). Homogenized organs were titrated 1:10 over 6 steps and layered over Vero cells. After 1 h of incubation at 37° C., a 1% methylcellulose in DMEM overlay was added, and the cells were incubated for 3 days at 37° C. Cells were fixed with 4% PFA and plaques were counted by crystal violet staining.

Pharmacokinetic Studies. Pharmacokinetic studies were conducted at Scripps Research Institute's Animal Models Core in accordance with IACUC guidelines (IACUC Protocol #09-0004-5). Eight-week old male Syrian Hamsters (Charles River) (three per group) were dosed PO as indicated for each compound and formulation. Plasma concentration of each test article was monitored up to 48 h. Nelfinavir was formulated in 10% DMSO/90% corn oil and MK-4482 was formulated in 10% PEG400/2.5% Cremaphor RH40 for both pharmacokinetic and efficacy studies.

Hamster Lung RNA Analysis. Hamster lung from uninfected (U, n=2), vehicle treated (V, n=4) and MK-4482 treated (T, n=4) samples were analyzed using RNASeq platform. Mean absolute deviation (MAD) is computed for all genes using python package scipy.stats.median_absolute_deviation. StepMiner algorithm (Sahoo, D., Dill, D. L., Tibshirani, R. & Plevritis, S. K. Extracting binary signals from microarray time-course data. Nucleic Acids Res 35, 3705-3712, doi:10.1093/nar/gkm284 (2007)) was applied to select the high MAD values which filter 22,284 genes down to 14,939. StepMiner algorithm was applied again to filter 14,939 down to 8,617 genes. Hierarchical agglomerative clustering analysis was performed on these 8,617 genes with python seaborn clustermap library function. Differential expression analysis is performed using DESeq227 (MK-4482 treated vs the vehicle treated samples) and adjusted pvalue<0.1 and |log 2 of the fold change|>1 is applied to identify up/down regulated genes. Reactome pathway analysis (Fabregat, A. et al. The Reactome Pathway Knowledgebase. Nucleic Acids Res 46, D649-D655, doi:10.1093/nar/gkx1132 (2018)) of differentially expressed genes was performed to identify the high-level the biological processes enriched in the gene set. A bar plot with −log 10(fdr) as x-axis is used to demonstrate the significance of the enriched biological processes.

RNASeq. RNA sequencing libraries were generated using the Illumina TruSeq Stranded Total RNA Library Prep Gold with TruSeq Unique Dual Indexes (Illumina, San Diego, CA) exactly as described before 25. Briefly, samples were processed following manufacturer's instructions, except modifying RNA shear time to five minutes. Resulting libraries were multiplexed and sequenced with 100 basepair (bp) Paired End (PE100) to a depth of approximately 25-40 million reads per sample on an Illumina NovaSeq 6000 by the Institute of Genomic Medicine (IGM) at the University of California San Diego. Samples were demultiplexed using bcl2fastq v2.20 Conversion Software (Illumina, San Diego, CA). RNASeq data was processed using kallisto (version 0.45.0), Mesocricetus anratus genome (MesAur1.0). Gene-level TPM values and gene annotations were computed using tximport and biomaRt R package. A custom python script was used to organize the data and log reduced using log 2(TPM) if TPM>1 and TPM−1 if TPM<=1. For the hamster study kallisto index was prepared on Mesocricetus_auratus.MesAur1.0.ncma.fa.gz+Mesocricetus_auratus MesAur1.0 cdna.all.fa.gz. The raw data and processed data are deposited in Gene Expression Omnibus (pending GSEID from NCBI GEO).

Hamster Lung Histopathology/Infiltrate Quantification. ImageJ software is used to quantify H&E stained slide images (at 20× magnification). Images are first converted to 8-Bit (Image>Type>8-Bit), their threshold is adjusted (Image>Adjust>Threshold), a threshold value between 70-80% is chosen to ensure only dark stained nuclei are detected. Following thresholding the image is converted to a mask (Process>Binary>Convert to Mask) and analyzed (Analyze>Analyze Particles) with default settings, adding display results and show outline and the output was exported into GraphPad Prism (V9.0.0) where the nonparametric, two-sided Mann-Whitney statistical test was used to calculate significance.

Data analysis. High-content image analysis was carried out with MetaXpress (version 6.5.4.532). Primary in vitro screen and the host cell cytotoxicity counter screen data were uploaded to Genedata Screener, Version 16.0.3-Standard. HeLa-ACE2 data were normalized to neutral (DMSO) minus inhibitor controls (2.4 μM remdesivir for antiviral effect in HeLa-ACE2 cells and 10 μM puromycin for infected host cell toxicity). Calu-3 infection assay data were normalized to neutral (DMSO) minus inhibitor control (10 μM remdesivir), and for the Calu-3 cell count readout the total cells were normalized to the stimulator (10 μM remdesivir) minus neutral control (DMSO). For the uninfected host cell cytotoxicity counter screens, 40 μM puromycin (Sigma) was used as the positive (inhibitory) control in HeLa-ACE2, HepG2 and HEK293T cells, and 30 μM puromycin (Sigma) was used as the positive (inhibitory) control for Calu-3 cells. For dose response experiments compounds were tested in technical triplicates on different assay plates and dose curves were fitted with the four parameter Hill Equation. Technical replicate data were analyzed using median condensing. Geometric means and geometric standard deviations are reported for compound activities (EC50s and CC50s) obtained in multiple independent biological experiments. The synergyfinder package in R (version 3.6.3) was used for synergy analysis (Ianevski, A., He, L., Aittokallio, T. & Tang, J. SynergyFinder: a web application for analyzing drug combination dose-response matrix data. Bioinformatics 33, 2413-2415, doi:10.1093/bioinformatics/btx162 (2017)). Geometric means were calculated by computing the logarithm (base 10) of all values, calculating the mean of these logarithms, and taking the antilog of that mean. Geometric standard deviations were computed by taking the standard deviation of the log-transformed individual values and taking the antilog of that standard deviation. The geometric standard deviation is a unitless ratio and reported as ×÷ instead of +/−. That is, fora reported 0.123 μM×÷1.276, the standard deviation range is from 0.096 μM to 0.157 μM (i.e. 0.123 μM÷1.276 to 0.123 μM×1.276).

High-throughput Calu-3 phenotypic ReFRAME screen against SARS-CoV-2. To complement the relatively rapid 24 h HeLa-ACE2 assay and prioritize hits, we developed a second, more physiologically-relevant infection assay using Calu-3 cells that relied on the same antibody detection and a similar assay workflow, with a readout at 48 hours post SARS-CoV-2 infection (hpi) (supra). Calu-3 are human lung epithelial cells that endogenously express both the ACE2 receptor and the host serine protease TMPRSS2, which is required for SARS-CoV-2 Spike protein processing and viral entry into host cells (Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.cell.2020.02.052 (2020)), while the robust infection in HeLa-ACE2 cells, which lack TMPRSS2 expression, is likely dependent on endosomal, cathepsin-mediated viral entry pathway that has been a generally recognized mechanism for coronaviruses (Yang, N. & Shen, H. M. Targeting the Endocytic Pathway and Autophagy Process as a Novel Therapeutic Strategy in COVID-19. Int J Biol Sci 16, 1724-1731, doi:10.7150/ijbs.45498 (2020)).

Remdesivir was active in Calu-3 cells (EC50=444 nM×÷1.514 (n=4)), as was the TMPRSS2 inhibitor nafamostat mesylate (EC50=24 nM×÷1.55 (n=3)). In contrast to the HeLa-ACE2 screen, cytopathic effect was more pronounced in the Calu-3 assay (likely due to higher MOI and longer incubation times used). As a result, antiviral compounds also protected the cells from virus-induced cell death, providing a second metric related to compound anti-viral activity. Notably, the majority of the 52 HeLa-ACE2 ReFRAME hits were either not active (58%, 30/52) or not selective in the Calu-3 cell-based assay.

Limited overlap in activities in HeLa-ACE2 and Calu-3 cells prompted a re-screen of the ReFRAME library using Calu-3 cells. The semen was carried out at a final concentration of 2.5 μM, RZ′=0.744, and there was identified 235 primary hits that demonstrated >50% inhibition of infection, <80% cell toxicity or >40% inhibition of infection and >40% increase in cell count (protection from virus-induced cell death). Of these, 145 were moderately active when tested in a dose-response format (EC50<10 μM), but only 42 were also selective (CC50/EC50>10 or CC50>30 μM). 88 putative hit compounds were chosen to test as fresh powder stocks (CC50/EC50>5 or CC50>30 μM, CC50/EC50<5 but with less than a 50% reduction in uninfected cytotoxicity assay, and 3 extra compounds with EC50<1 μM showing protection in infected cell count readout) and 87 reconfirmed as potent and 41 reconfirmed as also selective in Calu-3 cells. The 41 reconfirmed Calu-3 ReFRAME hits were likewise re-tested in the HeLa-ACE2 infection assay. Of these, 63% (26/41) were inactive against SARS-CoV-2 in HeLa-ACE2 cells, whereas 34% (14/41) were active but strongly cytotoxic, with a CC50<3 μM in uninfected HeLa-ACE2 cells. Identifying endosomal cathepsin-mediated entry inhibitors. As one likely source of limited activity of HeLa-ACE2 ReFRAME hits in the Calu-3 assay is the entry mechanism used by the virus in each cell line, we established a time of addition (TOA) assay in HeLa-ACE2 cells to identify cathepsin-mediated viral entry inhibitors among the ReFRAME hits, which are unlikely to be active in the context of TMPRSS2-entry. To first determine kinetics of infection, HeLa-ACE2 cells were infected for 1 h with SARS-CoV-2, after which un-adsorbed virus was washed off, and cells plated in 384-well plates in the presence of DMSO, hydroxychloroquine, apilimod, or remdesivir at a final concentration of 10 μM. Cells in wells were fixed as indicated, from 4 to 24 hpi and percent infected cells at each timepoint were quantified.

In all treatments except for remdesivir, viral infection was first apparent by antibody staining at 6 hpi and reached near maximal levels at 10 to 12 hpi. Loss of activity of both apilimod and hydroxychloroquine when treatment was initiated at 1 hpi indicates that these compounds block viral entry in HeLa-ACE2 cells while remdesivir treatment effectively blocked the infection, despite the initiation of treatment at 1 hpi, in line with its direct antiviral mechanism of action.

Based on these results, we used the 10 hpi timepoint to limit cycles of replication in a TOA assay (supra) in which we assessed the activity of all HeLa-ACE2 ReFRAME hits in dose response. We found that 33% (10/30) of compounds which were inactive in Calu-3 cells were entry inhibitors in HeLa-ACE2 based on the reduction of their activity in the TOA assay, i.e. an EC50 ratio of >10 between the standard 24 h and the TOA assay. In contrast, no compound that was also active in Calu-3 cells (EC50<10 μM) could as clearly be classified as an entry inhibitor at that threshold. Osimertinib and MK-2206 each had a ratio >8, suggesting they may be involved in viral entry in HeLa-ACE2 cells, however their antiviral activity in Calu-3 cells was unspecific (SI<2).

ReFRAME hit prioritization and validation. The ReFRAME library is a collection of bioactive small molecules, many of which are approved drugs or in clinical phases of development and used for a wide assortment of indications. The top five classes of potent and selective compounds reconfirmed as powders in the HeLa-ACE2 screen were oncolytic compounds (9), ion channel modulators (7), anti-inflammatory (5), antiviral (5) and signal transduction modulators (5), whereas in the Calu-3 screen the top five classes were signal transduction modulators (14), oncolytic compounds (11), protease inhibitors (7), antibiotics (3) and ion channel modulators (3). A fifth of the potent and selective hits in both screens could be classified as oncolytic drugs, reflecting the reliance of the virus on host cell processes present in rapidly proliferating cells. The identification of compounds belonging to anti-psychotic and anti-parasitic (neglected tropical diseases) classes exclusively in HeLa-ACE2 cells may reflect the cationic amphiphilic nature of some of these molecules and their ability to accumulate in and impact acidic intracellular compartments (e.g. late endosomes/lysosomes). Resultant dysregulation of the endo-lysosomal pathway and lipid homeostasis has been suggested to impair viral entry and/or replication (Salata, C., Calistri, A., Parolin, C., Baritussio, A. & Palu, G. Antiviral activity of cationic amphiphilic drugs. Expert Rev Anti Infect Ther 15, 483492, doi:10.1080/14787210.2017.1305888 (2017)), and this mode of action has been speculated for amiodarone and hydroxychloroquine, both identified as potent and selective hits against SARS-CoV-2 in the HeLa-ACE2 screen. However, only hydroxychloroquine was identified as an entry inhibitor in our assay.

From compounds identified as hits in our primary screens of high interest were compounds with a profile like that of remdesivir, which were active and selective in both HeLa-ACE2 and Calu-3 assays and were not classified as entry inhibitors in HeLa-ACE2 cells. The parent of prodrug MK-4482. N-hydroxycytidine matched that profile, although MK-4482 was itself not active in vitro, likely due to lack of metabolism that would turn it over to its active form.

Additionally, compounds such as nafamostat mesylate, the TMPRSS2 inhibitor, active in Calu-3 but not active in HeLa-ACE2 cells had the potential to be active in advanced models of infection. Conversely, entry inhibitors in HeLa-ACE2 cells that are not active in Calu-3 cells (e.g. apilimod, hydroxychloroquine, azithromycin) were deprioritized. Based on our prioritization, we tested activity of representative hits against SARS-CoV-2 in an orthogonal air-liquid interface primary human bronchial epithelial cell (ALI-HBEC) model of infection. These differentiated airway cells express high levels of both ACE2 and TMPRSS2. As expected, remdesivir and nafamostat mesylate inhibited viral replication in ALI-HBECs, while apilimod did not. Furthermore, nelfinavir mesylate, MK-4482 and its parent N-hydroxycytidine all caused a >1-log reduction in apical viral loads at 72 hpi. These results agreed with our model of hit prioritization.

Overall, we identified approved oral drugs halofantrine HCl, nelfinavir mesylate, simeprevir, and manidipine as hits of highest interest due to their activity in both assays and their relatively high exposures or a long history of use as therapeutic agents and therefore potential to be quickly repurposed as COVID-19 treatments following further efficacy vetting in animal models. The viral protease inhibitors nelfinavir and simeprevir have reported good plasma exposures and based on their described mode of action they may inhibit SARS-CoV-2 directly. The approved calcium-channel blocker manidipine has low plasma exposure but may have the potential to improve COVID-19 disease outcomes for patients. Nine other compounds in various stages of development also have high likelihood to show efficacy due to their potency in the screening assays or pharmacokinetic profiles (see tables). TO-195 and RWJ-56423 are both trypsin inhibitors and avoralstat is a kallikrein inhibitor active in Calu-3 cells which may block viral entry. The p38 mitogen-activated protein kinase (MAPK) inhibitor. LY222820/Ralimetinib mesylate, was active in both HeLa-ACE2 and Calu-3 screens and was previously shown to inhibit replication of other coronaviruses via inhibition of p38 MAPK23. Thus, p38 MAPK may be an important host target for inhibiting coronavirus replication. Of note, N-hydroxycytidine, the parent of the prodrug MK-4482 (Molnupiravir, EIDD-2801) was a potent and selective hit in both the HeLa-ACE2 and Calu-3 assays. MK-4482 is an oral antiviral nucleoside analogue currently being evaluated by Ridgeback Biotherapeutics and Merck in treatment of COVID-19 patients.

MK-4482 oral dosing is fully protective against SARS-CoV-2-infection. Due to the demonstrated in vitro potency in the ALI-HBEC primary cell model and adequate exposures of nelfinavir and MK-4482/N-hydroxycytidine (a time over Calu-3 SARS-CoV-2 EC50 of ˜3 h for a single 500 mg/kg PO dose of nelfinavir, and time over HeLa-ACE2 and Calu-3 EC50≥7 h for a single 500 mg/kg PO dose of MK-4482), we investigated the efficacy of nelfinavir and MK-4482 in a Golden Syrian hamster animal model of SARS-CoV-2 infection. Nelfinavir was delivered PO at 500 mg/kg BID (twice daily) and MK-4482 was delivered PO at 500 mg/kg, 150 mg/kg, and 50 mg/kg BID, to evaluate dose-dependent protection. A matched vehicle-only suspension was used as a control. Four hours after first treatment, animals were challenged with 1×106 PFU of SARS-CoV-2 (USA-WA1/2020) by intranasal administration. The animals were weighed daily as a measure of disease progression and lung tissue was isolated on day five of infection to determine viral titers, lung histology and gene expression profiles. Nelfinavir failed to protect animals from weight loss and viral replication, potentially due to inadequate plasma exposure in hamsters. However, MK-4482 protected animals from severe weight loss at 500 mg/kg, averaging 97% of their starting weight at day 5 of infection. The 150 mg/kg and 50 mg/kg groups showed partial protection through weight loss, averaging 89% and 90% of their starting weight, respectively, compared to the vehicle control 85% at day 5 of infection.

To analyze correlations to weight loss, the relative virus titers were determined from day five lung samples using a crystal violet-based plaque assay. The 500 mg/kg and 150 mg/kg doses had undetectable live viral titers in the lungs, showing full protection from virus replication. The 50 mg/kg group averaged 4.5×103 PFU/lung, showing moderately good efficacy (99% viral reduction) compared to the vehicle control group which averaged 4.5×105 PFU/lung.

Protection from weight loss and viremia in the 500 mg/kg treatment arm was associated with a near-complete protection from host immune response, as determined by RNA Seq analysis on hamster lungs followed by unsupervised hierarchical clustering: the MK-4482-treated (500 mg/kg) samples clustered together with uninfected samples. A DESeq2 analysis confirmed that infected vehicle-treated lungs induce the expression of 66 genes associated with pathways reported to be upregulated in COVID-19, including interferon signaling and interferon stimulated genes (Tindle, C. et al. Adult Stem Cell-derived Complete Lung Organoid Models Emulate Lung Disease in COVID-19. bioRxiv, doi:10.1101/2020.10.17.344002 (2020); Sahoo, D. et al. Al-guided discovery of the invariant host response to viral pandemics. bioRxiv, doi:10.1101/2020.09.21.305698 (2020)). Finally, histological examination confirmed that the lungs from MK-4482-treated hamsters were protected and more closely resembled those tissues from uninfected animals. In stark contrast, examination of lung tissue in the vehicle-treated control group revealed obliteration of alveolar spaces and overwhelming immune cell infiltration.

### NUMBERED REFERENCES CITED IN THIS DISCLOSURE

1. A. Zumla, J. F. Chan, E. I. Azhar, D. S. Hui, K. Y. Yuen. Coronaviruses—drug discovery and therapeutic options. Nat Rev Drug Discov 15, 327-347 (2016).
2. E. de Wit et al., Prophylactic and therapeutic remdesivir (GS-5734) treatment in the rhesus macaque model of MERS-CoV infection. Proc Natl Acad Sci USA 117, 6771-6776 (2020).
3. T. P. Sheahan et al., Broad-spectrum antiviral GS-5734 inhibits both epidemic and zoonotic coronaviruses. Sci Transl Med 9, (2017).
4. M. K. Lo et al., GS-5734 and its parent nucleoside analog inhibit Filo-, Pneumo-, and Paramyxoviruses. Sci Rep 7, 43395 (2017).
5. J. Janes et al., The ReFRAME library as a comprehensive drug repurposing library and its application to the treatment of cryptosporidiosis. Proc Natl Acad Sci USA 115, 10750-10755 (2018).
6. M. Wang et al., Remdesivir and chloroquine effectively inhibit the recently emerged novel coronavirus (2019-nCoV) in vitro. Cell Res 30, 269-271 (2020).
7. M. Prajapat et al., Drug targets for corona virus: A systematic review. Indian J Pharmacol 52, 56-65 (2020).
8. C. Salata, A. Calistri, C. Parolin, A. Baritussio, G. Palu, Antiviral activity of cationic amphiphilic drugs. Expert Rev Anti Infect Ther 15, 483-492 (2017).
9. L. M. Johansen et al., FDA-approved selective estrogen receptor modulators inhibit Ebola virus infection. Sci Transl Med 5, 190ra179 (2013).
10. M. B. Oldstone, J. R. Teijaro, K. B. Walsh, H. Rosen, Dissecting influenza virus pathogenesis uncovers a novel chemical approach to combat the infection. Virology 435, 92-101 (2013).
11. M. Mazzon et al. Identification of Broad-Spectrum Antiviral Compounds by Targeting Viral Entry. Viruses 11, (2019).
12. E. A. Nelson et al., The phosphatidylinositol-3-phosphate 5-kinase inhibitor apilimod blocks filoviral entry and infection. PLoS Negl Trop Dis 11, e0005540 (2017).
13. A. Ianevski, L. He, T. Aittokallio, J. Tang, SynergyFinder: a web application for analyzing drug combination dose-response matrix data. Bioinformatics 33, 2413-2415 (2017).
14. B. Yadav, K. Wennerberg, T. Aittokallio, J. Tang, Searching for Drug Synergy in Complex Dose-Response Landscapes Using an Interaction Potency Model. Comput Struct Biotechnol J 13, 504-513 (2015).
15. Summary on compassionate use Remdesivir Gilead Procedure No. EMEA/H/K/5622/CU (EMA/178637/2020).
16. C. J. Porter et al., Use of in vitro lipid digestion data to explain the in vivo performance of triglyceride-based oral lipid formulations of poorly water-soluble drugs: studies with halofantrine. J Pharm Sci 93, 1110-1121 (2004).
17. K. A. Milton, G. Edwards, S. A. Ward, M. L. Orme, A. M. Breckenridge, Pharmacokinetics of halofantrine in man: effects of food and dose size. Br J Clin Pharmacol 28, 71-77 (1989).
18. S. Morita, T. Takahashi, Y. Yoshida. N. Yokota, Population Pharmacokinetics of Hydroxychloroquine in Japanese Patients With Cutaneous or Systemic Lupus Erythematosus. Ther Drug Monit 38, 259-267 (2016).
19. J. Emami, Comparative in vitro and in vivo evaluation of three tablet formulations of amiodarone in healthy subjects. Daru 18, 193-199 (2010).
20. I. Pellegrin et al., Virologic response to nelfinavir-based regimens: pharmacokinetics and drug resistance mutations (VIRAPHAR study). AIDS 16, 1331-1340 (2002).
21. J. Snoeys. M. Beumont, M. Monshouwer, S. Ouwerkerk-Mahadevan, Mechanistic understanding of the nonlinear pharmacokinetics and intersubject variability of simeprevir: A PBPK-guided drug development approach. Clin Pharmacol Ther 99, 224-234 (2016).
22. H. W. Reesink et al., Rapid HCV-RNA decline with once daily TMC435: a phase I study in healthy volunteers and hepatitis C patients. Gastroenterology 138, 913-921 (2010).
23. A. Stockis et al., Pharmacokinetics and tolerability of a new manidipine and delapril fixed oral combination in young and elderly subjects. Arzneimittelforschung 53, 554-561 (2003).
24. J. Q. Tran et al., Results From the First-in-Human Study With Ozanimod, a Novel, Selective Sphingosine-1-Phosphate Receptor Modulator. J Clin Pharmacol 57, 988-996 (2017).
25. N. Strojny, J. A. de Silva, Determination of diclofensine, an antidepressant agent, and its major metabolites in human plasma by high-performance liquid chromatography with fluorometric detection. J Chromatogr 341, 313-331 (1985).
26. P. Salva. J. Costa. A. Perez-Campos, A. Martinez-Tobed, Repeated dose pharmacokinetics of pancopride in human volunteers. Biopharm Drug Dispos 15, 643-651 (1994).
27. D. C. S. Harb Wael A, Lakhani Nehal, Rutherford Sarah C, Schreeder Marshall T, Ansell Stephen M, Sher Taimur, Aboulafia David M, Cohen Jonathon B, Nix Darrell, Landrette Sean, Flanders Kate, Miller Langdon L. Lichenstein Henri, Abramso Jeremy S, Phase 1 Clinical Safety, Pharmacokinetics (PK), and Activity of Apilimod Dimesylate (LAM-002A), a First-in-Class Inhibitor of Phosphatidylinositol-3-Phosphate 5-Kinase (PIKfyve), in Patients with Relapsed or Refractory B-Cell Malignancies. ASH Annual Meeting 2017 Dec. 9-12 Atlanta, GA, USA.
28. A. Patnaik et al., A First-in-Human Phase I Study of the Oral p38 MAPK Inhibitor, Ralimetinib (LY2228820 Dimesylate), in Patients with Advanced Cancer. Clin Cancer Res 22, 1095-1102 (2016).
29. T. Doi et al., Phase 1 pharmacokinetic study of the oral pan-AKT inhibitor MK-2206 in Japanese patients with advanced solid tumors. Cancer Chemother Pharmacol 76, 409-416 (2015).
30. T. C. Wnuk-Lipinska Katarzyna, Gausdal Gro, Sandal Tone, Frink Robin, Hinz Stefan, Hellesoy Monica. Ahmed Lavina. Haugen Hallvard, Liang Xiao, Blo Magnus, Micklem David, Yule Murray, Minna John, Zhou Longen, Brekken Rolf. Lorens James. BGB324, a selective small molecule Axl kinase inhibitor to overcome EMT-associated drug resistance in carcinomas: Therapeutic rationale and early clinical studies. Proceedings of the 105th Annual Meeting of the American Association for Cancer Research 2014 Apr. 5-9, (2014).
31. N. Song, S. Zhang. Q. Li. C. Liu, Establishment of a liquid chromatographic/mass spectrometry method for quantification of tetrandrine in rat plasma and its application to pharmacokinetic study. J Pharm Biomed Anal 48, 974-979 (2008).
32. R. A. Steams et al., The pharmacokinetics of a thiazole benzenesulfonamide beta 3-adrenergic receptor agonist and its analogs in rats, dogs, and monkeys: improving oral bioavailability. Drug Metab Dispos 30, 771-777 (2002).
33. K. Kiura et al., Osimertinib in patients with epidermal growth factor receptor T790M advanced non-small cell lung cancer selected using cytology samples. Cancer Sci 109, 1177-1184 (2018).
34. H. Zhao et al., Pharmacokinetics of Osimertinib in Chinese Patients With Advanced NSCLC: A Phase 1 Study. J Clin Pharmacol 58, 504-513 (2018).
35. N. A. Naryshkin et al., Motor neuron disease. SMN2 splicing modifiers improve motor function and longevity in mice with spinal muscular atrophy. Science 345, 688-693 (2014).
36. L. Patel et al., Discovery of Orally Efficacious Phosphoinositide 3-Kinase delta Inhibitors with Improved Metabolic Stability. J Med Chem 59, 9228-9242 (2016).
37. W. N. Washburn et al., Identification of a nonbasic melanin hormone receptor 1 antagonist as an antiobesity clinical candidate. J Med Chem 57, 7509-7522 (2014).
38. D. L. Hertzog et al., The discovery and optimization of pyrimidinone-containing MCH R1 antagonists. Bioorg Med Chem Lett 16, 4723-4727 (2006).
39. D. C. Cole et al., Discovery of N1-(6-chloroimidazo[2,1-b][1,3]thiazole-5-sulfonyl)tryptamine as a potent, selective, and orally active 5-HT(6) receptor agonist. J Med Chem 50, 5535-5538 (2007).

### INCORPORATION BY REFERENCE

All publications, patents, and patent applications mentioned in this specification are incorporated by reference herein to the same extent as if each individual publication, patent, or patent application was specifically and individually indicated to be incorporated by reference.

We claim:

1. A method for treating a subject having an infection by a pathogen, comprising administering to the subject a therapeutically effective amount of at least one compound selected from the following table:

| Name | Structure |
|---|---|
| RFM-011-200-5 | 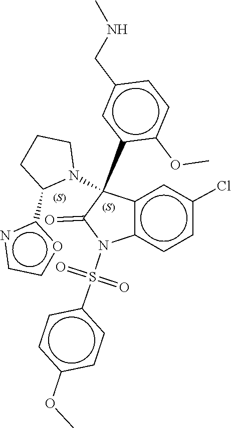 |
| RFM-007-454-4 | 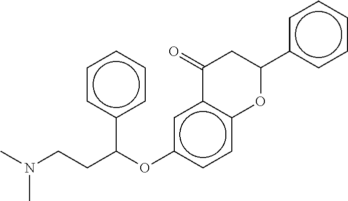 |
| bazedoxifene acetate | 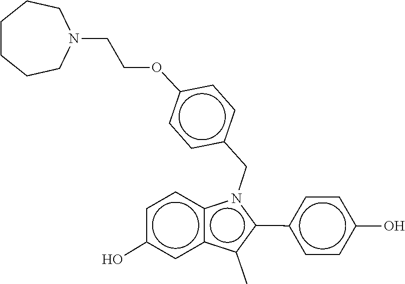 |
| Asenapine maleate | 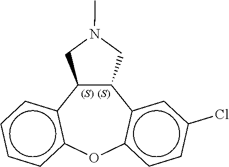 |
| manidipine | 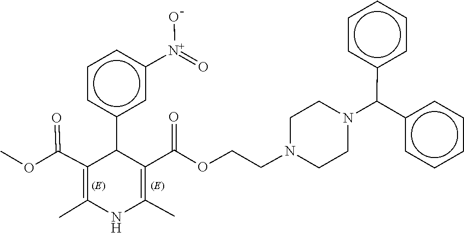 |
| Trimipramine | 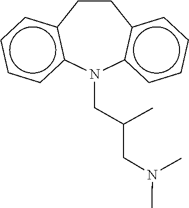 |
| Hanfangchin A | 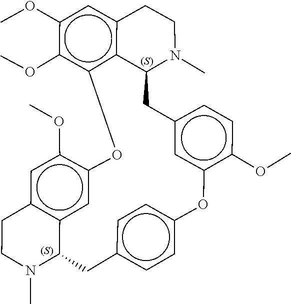 |
| Dabigatran etexilate mesilate | 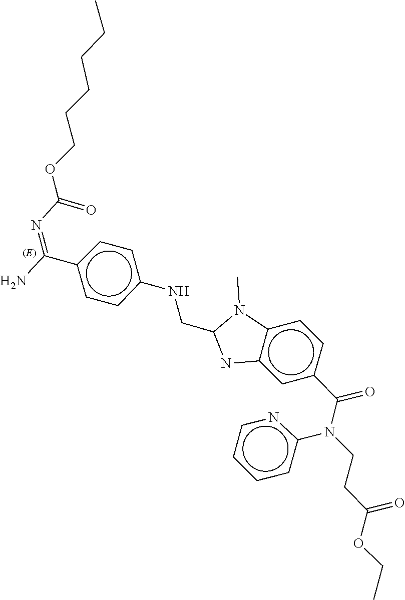 |
| Boceprevir | 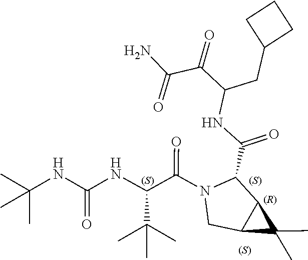 |
| nebivolol hydrochloride | 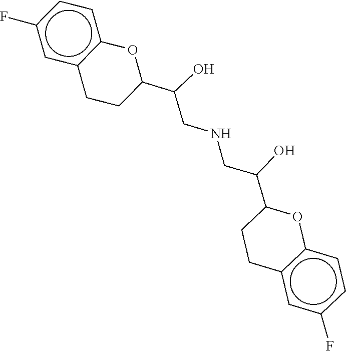 |
| Sorafenib | 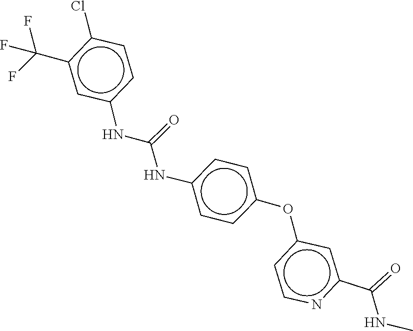 |
| Cepharanthine | 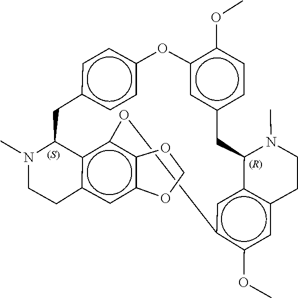 |
| raloxifene hydrochloride | 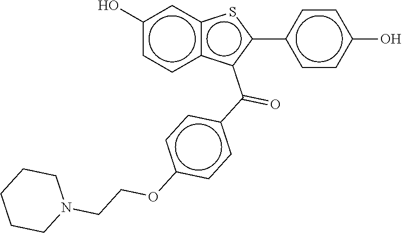 |
| AZD-5363 | 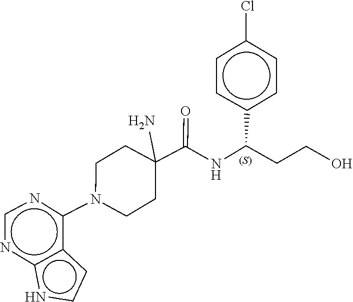 |
| Ponatinib | 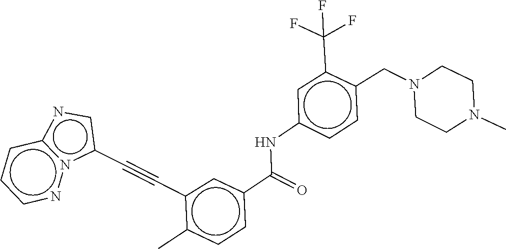 |
| MK-2206 | 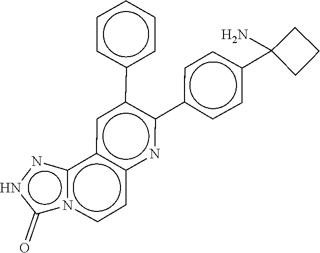 |
| Ralimetinib mesylate/LY2228820 | 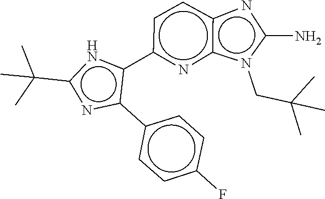 |
| Reserpine | 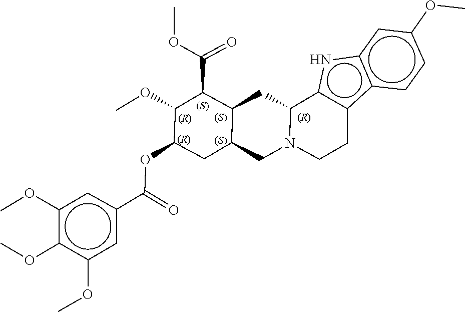 |
| amiodarone | 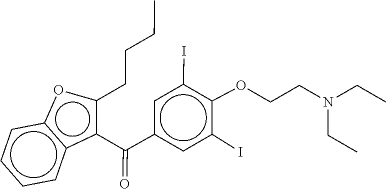 |
| Thioproperazine | 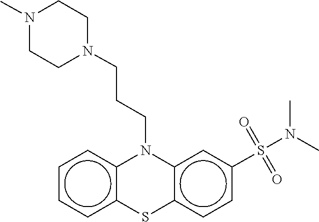 |
| Digoxin | 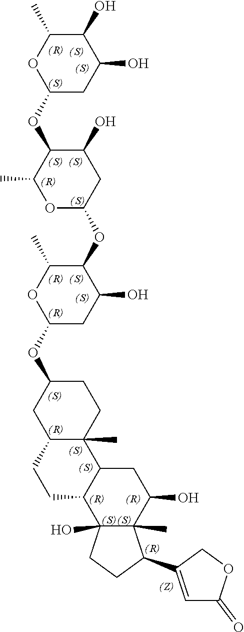 |
| BGB324/R-428 | 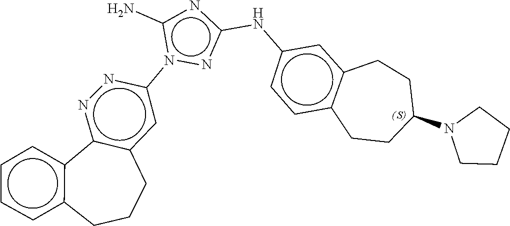 |
| nelfinavir mesylate | 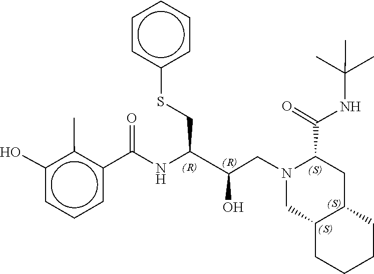 |
| TILORINE DIHYDROCHLORIDE | 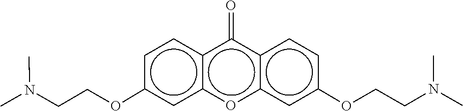 |
| Ozanimod | 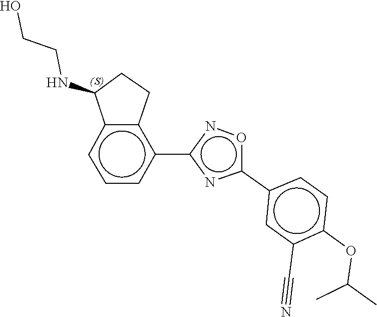 |
| APY0201 | 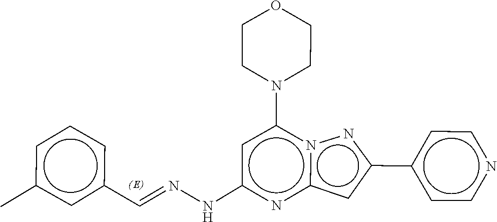 |
| GW-803430 | 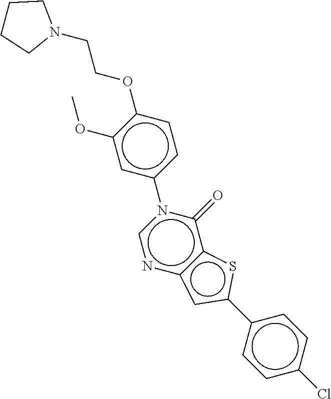 |
| ABT-239 | 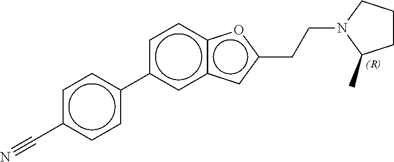 |
| pyronaridine | 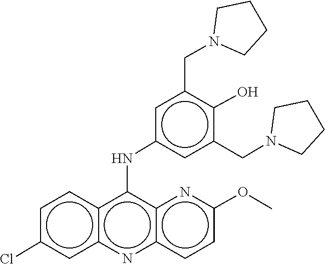 |
| R-7112 | 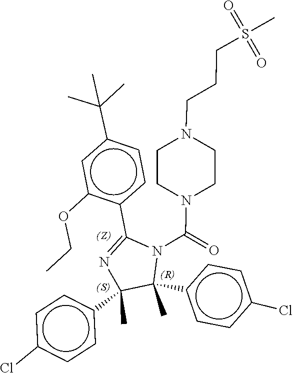 |
| Simeprevir | 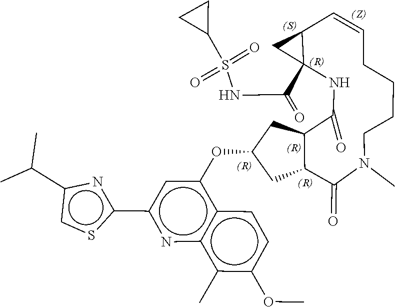 |
| OCTOCLOTHEPIN analog | 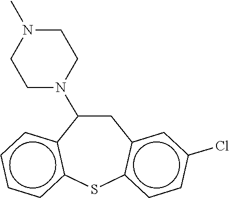 |
| Thalicarpine Analog | 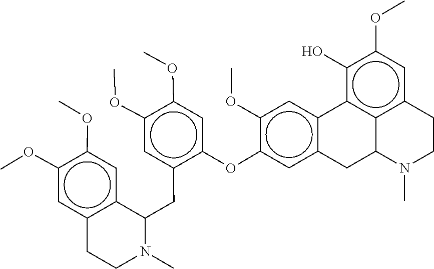 |
| SAX-187 | 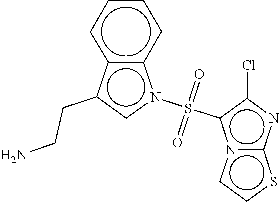 |
| MP-412 | 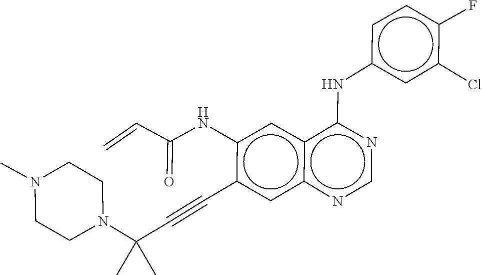 |
| 8-Chloroadenosine | 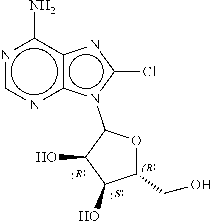 |
| DESMETHYLASTE MIZOLE | 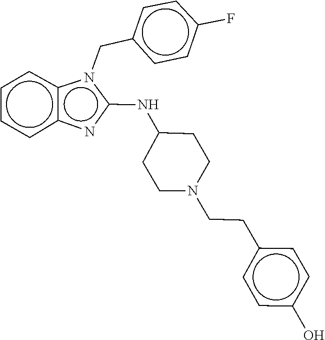 |
| AHR-5333 | 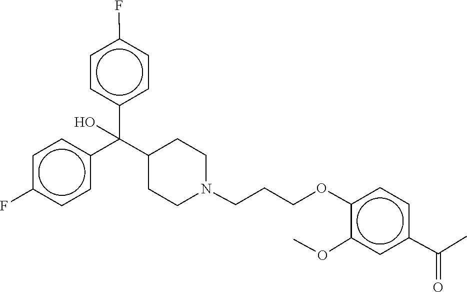 |
| Osimertinib | 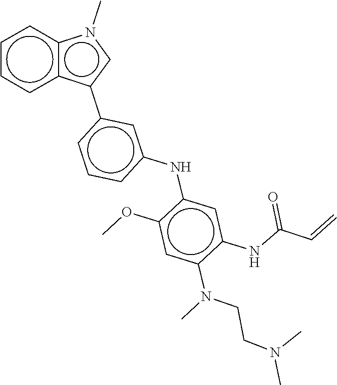 |
| PIPAMAZINE | 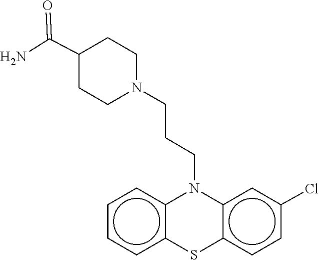 |
| AQ-13 | 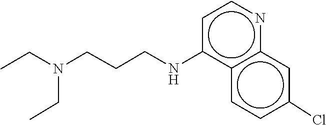 |
| YM-75440 | 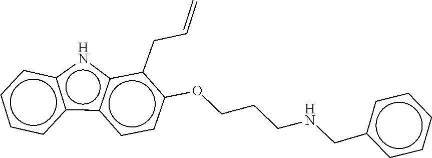 |
| Alkene Stereoisomer analog of Rilapine | 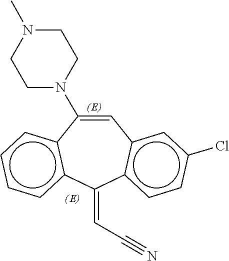 |
| Analog of Centbucridine | 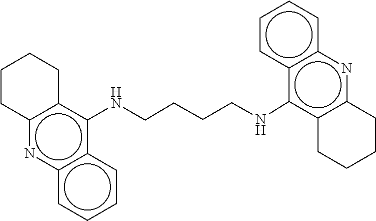 |
| tesevatinib | 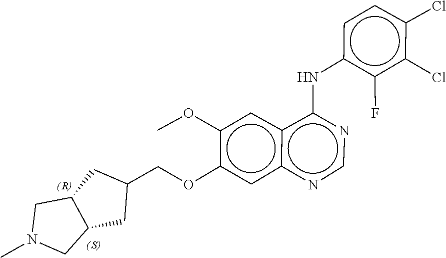 |
| Ferroquine | 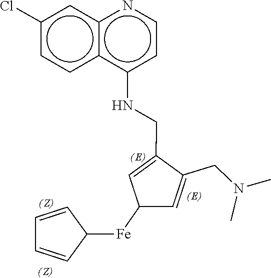 |
| TAK-070 | 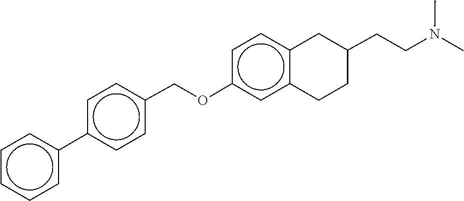 |
| SMN-C3 | 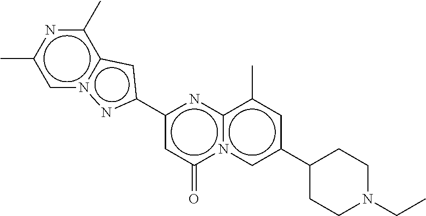 |
| LG-6-101 | 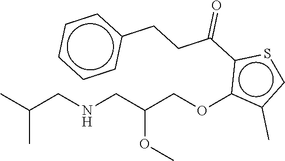 |
| CFI-400945 | 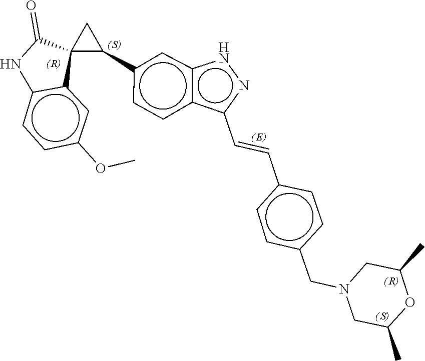 |
| NVX-207 | 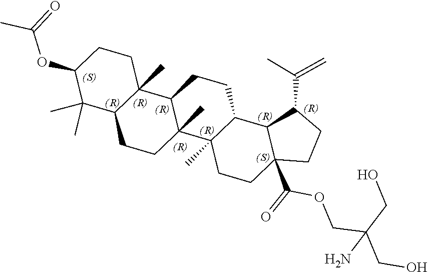 |
| KC 11404 | 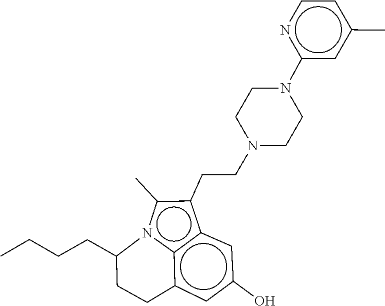 |
| NNC 090026 | 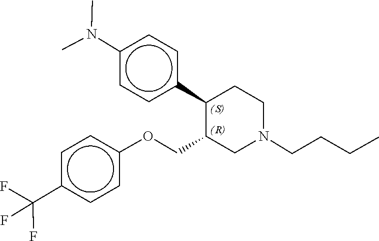 |
| CR-3124 | 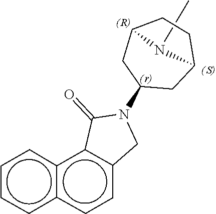 |
| ZUCLPENTHIXL | 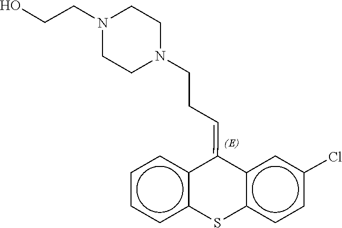 |
| R-116301 | 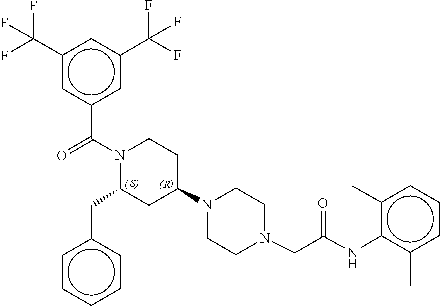 |
| ACOLBIFENE | 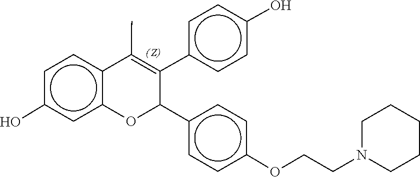 |
| Thalicarpine | 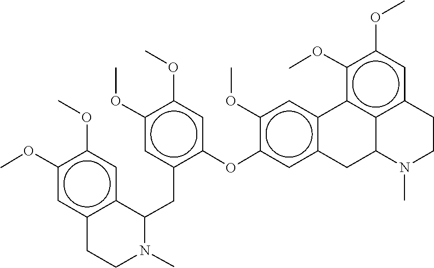 |
| YM 430 | 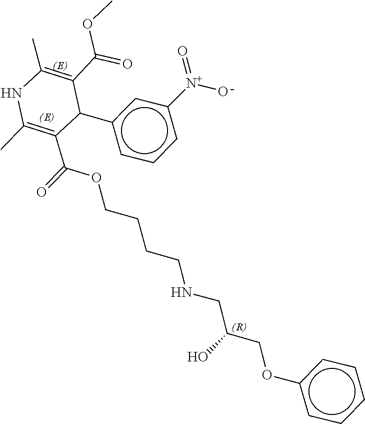 |
| NCO 700 | 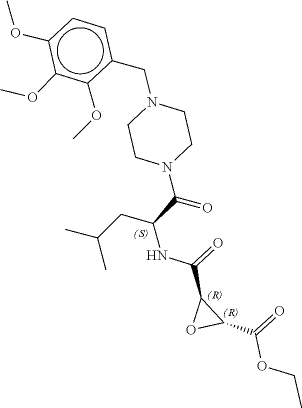 |
| Dutacatib | 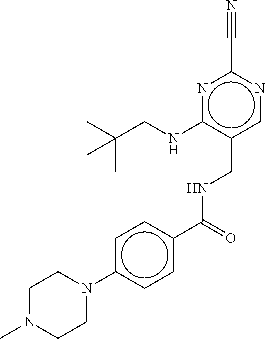 |
| M-55532 | 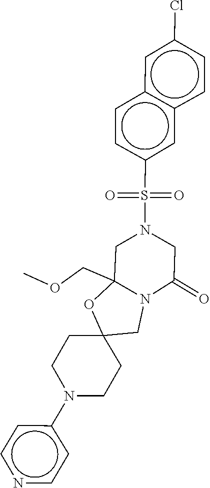 |
| YM 161514 | 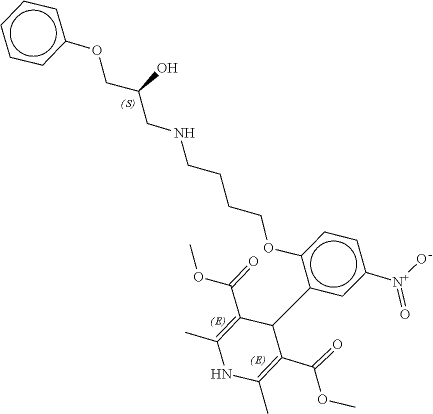 |
| Integrity ID 725781 | 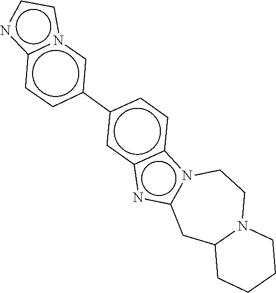 |
| MONATEPIL | 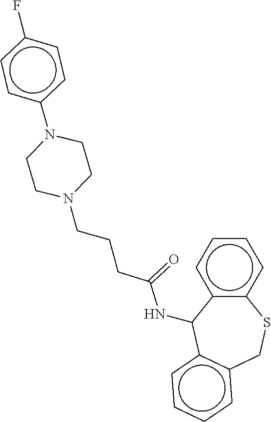 |
| Pancopride | 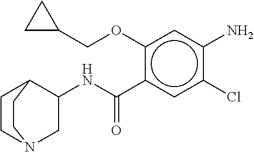 |
| Halofantrine HCl | 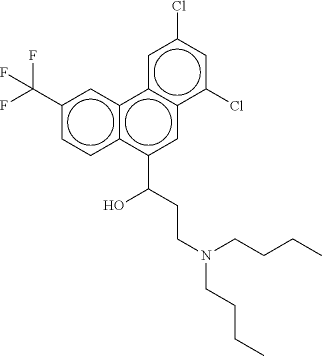 |
| AMOPYROQUINE | 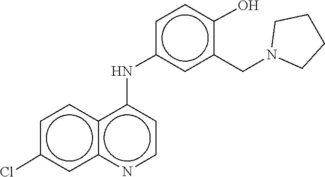 |
| A 81834 | 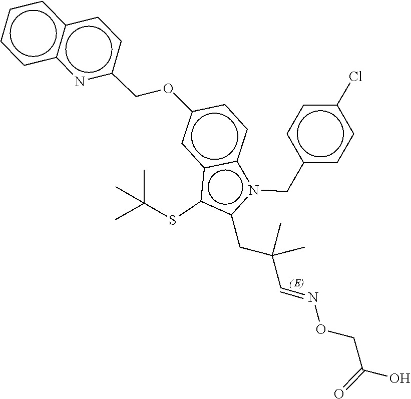 |
| L 796568 | 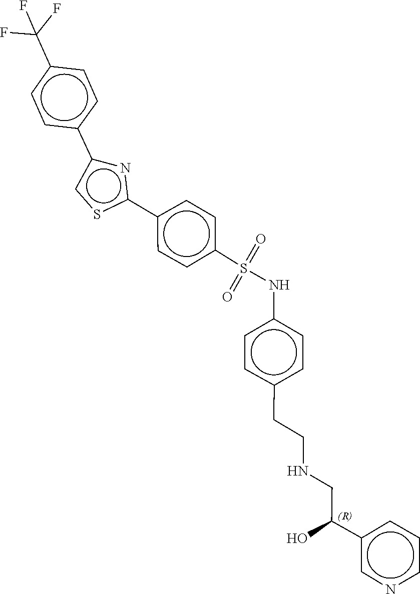 |
| ROPITOIN | 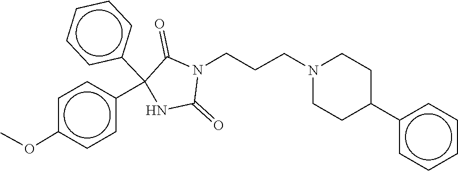 |
| S-33084 | 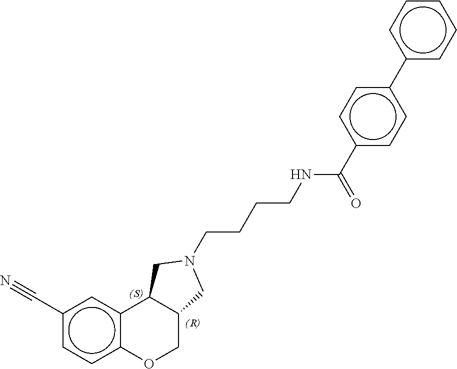 |
| RFM-011-761-3 | 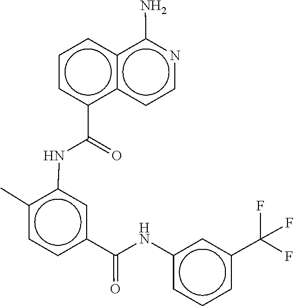 |
| Narasin | 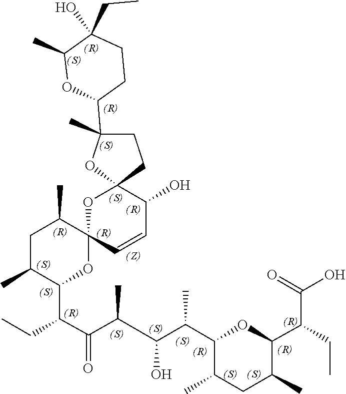 |
| Ethylisobutrazine Hydrochloride | 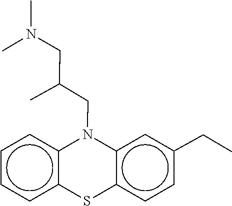 |
| GS-9901 | 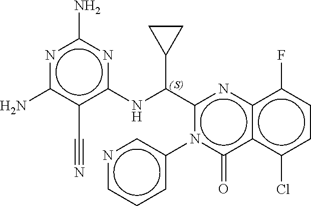 |
| Des-ethyl human metabolite of amodiaquine | 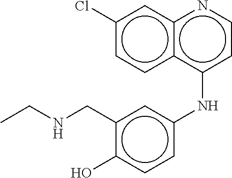 |
| Mequitazine | 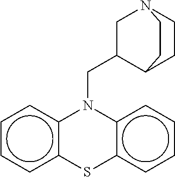 |
| Rupinavir | 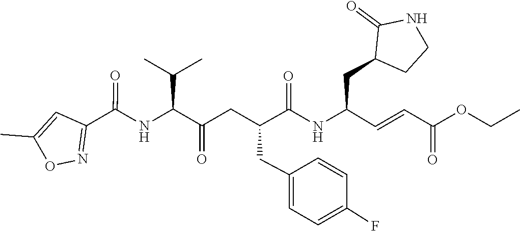 [SMILES: CCOC(=O)/C=C/[C@H](C[C@@H]1CCNC1=O)NC(=O)[C@@H](CC(=O)[C@@H](NC(=O)c1cc(C)on1)C(C)C)Cc1ccc(F)cc1] |
| albaconazole | 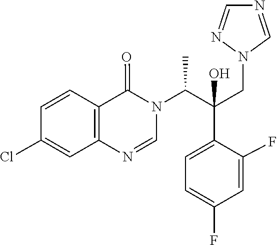 [SMILES: C[C@@H](n1cnc2cc(Cl)ccc2c1=O)[C@](O)(Cn1cncn1)c1ccc(F)cc1F] |
| Balicatib | 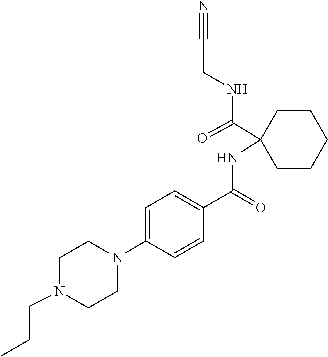 [SMILES: CCCN1CCN(c2ccc(C(=O)NC3(C(=O)NCC#N)CCCCC3)cc2)CC1] |
| Risdiplam | 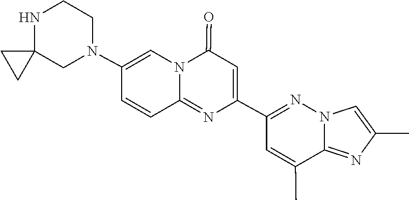 [SMILES: Cc1cn2nc(-c3cc(=O)n4cc(N5CCNC6(CC6)C5)ccc4n3)cc(C)c2n1] |
| Ebselen | 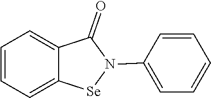 [SMILES: O=c1c2ccccc2[se]n1-c1ccccc1] |
| Arbidol | 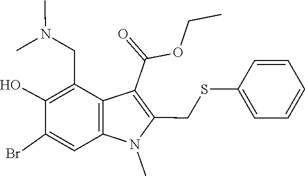 [SMILES: CCOC(=O)c1c(CSc2ccccc2)n(C)c2cc(Br)c(O)c(CN(C)C)c12] |
| Z LVG CHN2 | 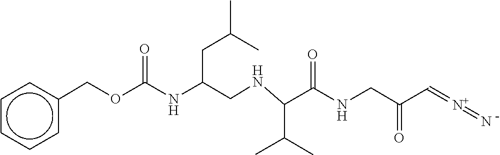 |
| Amodioaquine | 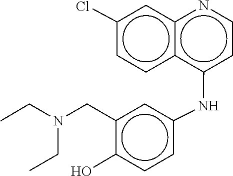 |
| Imatinib mesylate | 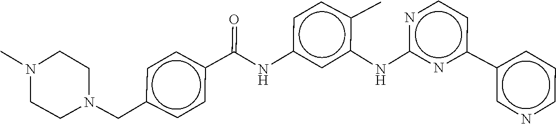 |

2. A method for treating a subject having an infection by a pathogen, comprising administering to the subject a therapeutically effective amount of at least one compound selected from the following table:

| Name | Structure |
|---|---|
| RWJ-56423 | 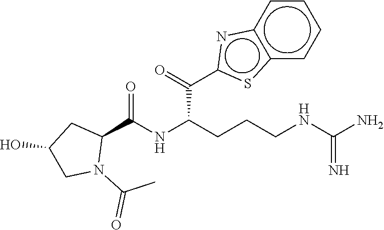 |
| TO-195 | 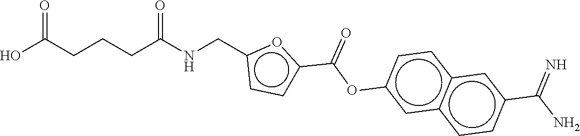 |
| Avoralstat | 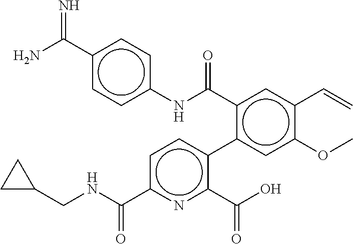 |
| YM 60828 | 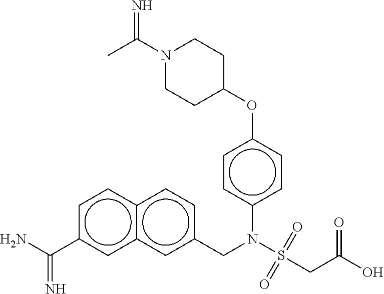 |
| UK-356202 | 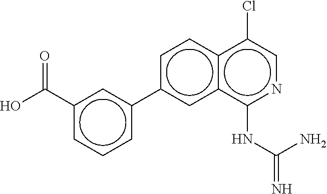 |
| Bardoxolone Methyl | 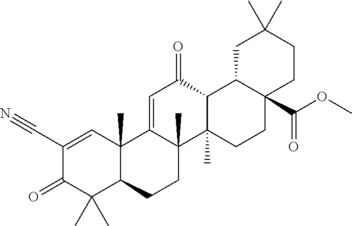 |
| MK-8722 | 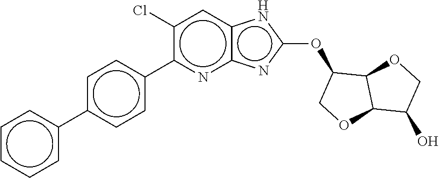 |
| AZ-11713908 | 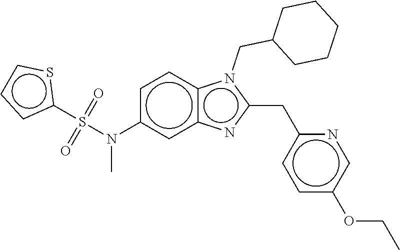 |
| cerivastatin (sodium salt) | 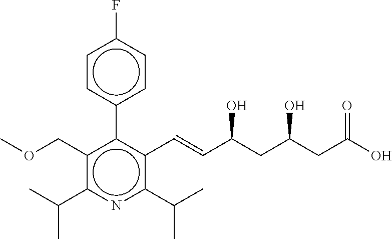 |
| CYMARINE | 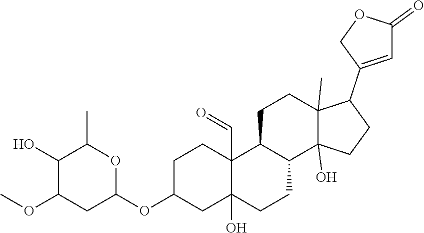 |
| Ono-3307 | 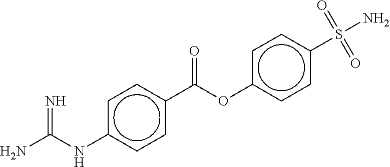 |
| AMA-0076 | 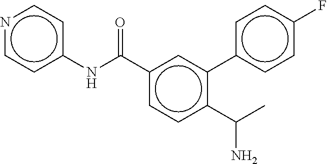 |
| Mitoguazone | 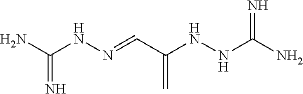 |
| Resiniferatoxin | 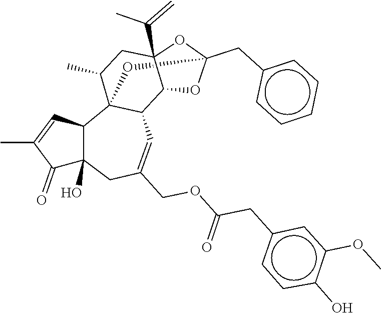 |
| DEAZANEPLANOCIN A | 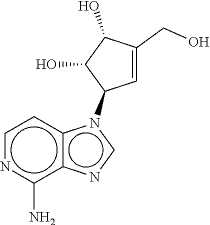 |
| OLIGOMYCIN A | 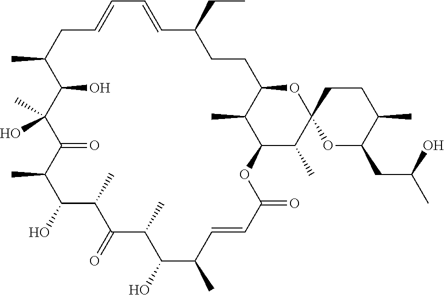 [SMILES: CC[C@@H]1/C=C/C=C/C[C@H](C)[C@@H](O)[C@](C)(O)C(=O)[C@H](C)[C@@H](O)[C@H](C)C(=O)[C@H](C)[C@@H](O)[C@H](C)/C=C/C(=O)O[C@H]2[C@@H](C)[C@@H](CC1)O[C@@]1(CC[C@@H](C)[C@@H](C[C@H](C)O)O1)[C@@H]2C] |
| Oligomycin B | 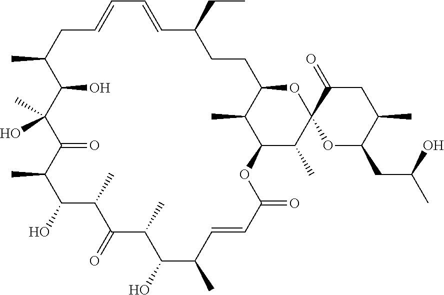 [SMILES: CC[C@@H]1/C=C/C=C/C[C@H](C)[C@@H](O)[C@](C)(O)C(=O)[C@H](C)[C@@H](O)[C@H](C)C(=O)[C@H](C)[C@@H](O)[C@H](C)/C=C/C(=O)O[C@H]2[C@@H](C)[C@@H](CC1)O[C@]1(O[C@H](C[C@H](C)O)[C@H](C)CC1=O)[C@@H]2C] |
| BMS-223131 | 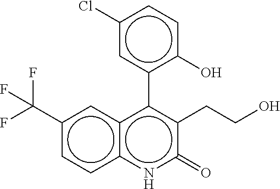 |
| Lestaurtinib | 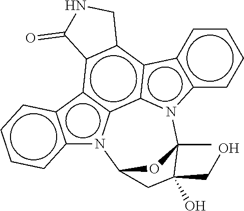 |
| Gemcitabine elaidate | 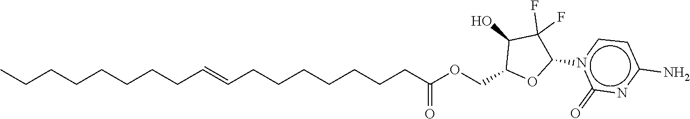 |
| Bruceantin | 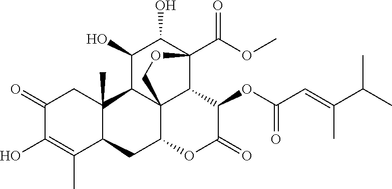 [SMILES: COC(=O)[C@@]12OC[C@]34[C@H]([C@@H](O)[C@@H]1O)[C@@]1(C)CC(=O)C(O)=C(C)[C@@H]1C[C@H]3OC(=O)[C@H](OC(=O)/C=C(\C)C(C)C)[C@@H]24] |
| LANATOSIDE C | 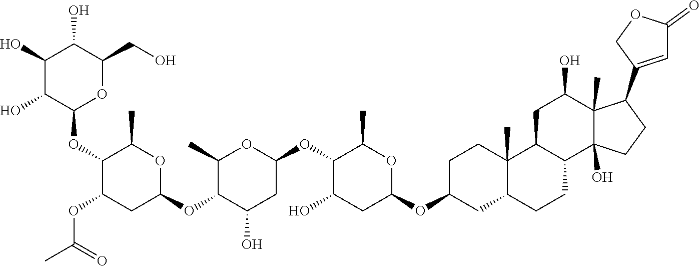 [SMILES: CC(=O)O[C@H]1C[C@H](O[C@H]2[C@@H](O)C[C@H](O[C@H]3[C@@H](O)C[C@H](O[C@H]4CC[C@@]5(C)[C@H](CC[C@@H]6[C@@H]5C[C@@H](O)[C@]5(C)[C@@H](C7=CC(=O)OC7)CC[C@]65O)C4)O[C@@H]3C)O[C@@H]2C)O[C@H](C)[C@H]1O[C@@H]1O[C@H](CO)[C@@H](O)[C@H](O)[C@H]1O] |
| ANISOMYCIN | 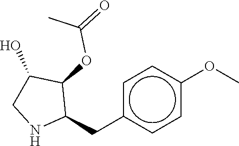 |
| Cephaeline | 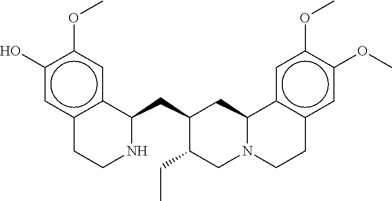 |
| SR-26050 | 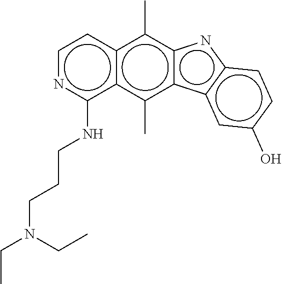 |
| Emetine | 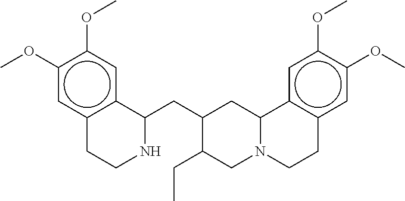 |
| Phorbol 12- myristate 13- acetate | 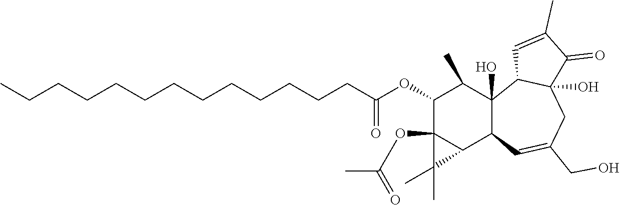 [SMILES: CCCCCCCCCCCCCC(=O)O[C@@H]1[C@@H](C)[C@@]2(O)[C@@H](C=C(CO)C[C@]3(O)C(=O)C(C)=C[C@@H]23)[C@@H]2C(C)(C)[C@]12OC(C)=O] |
| ARN-810 | 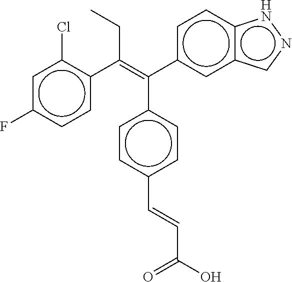 |
| VX-803 | 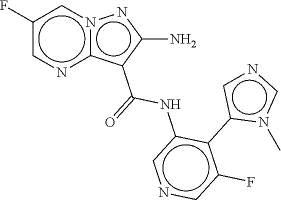 |
| E-7090 | 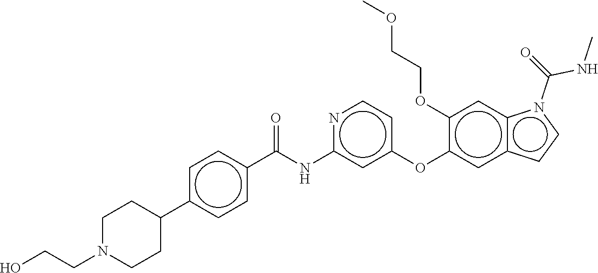 |
| Verosudil | 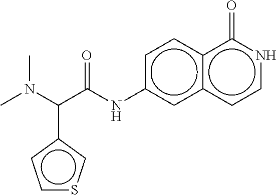 |
| Cycloheximide | 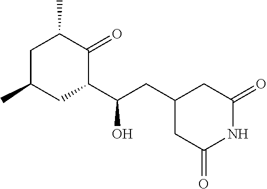 [SMILES: C[C@@H]1C[C@@H]([C@H](O)CC2CC(=O)NC(=O)C2)C(=O)[C@@H](C)C1] |
| PYRIDABEN | 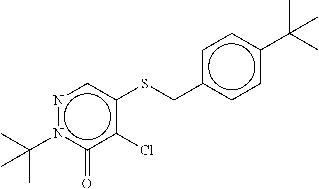 |
| Antimycin A | 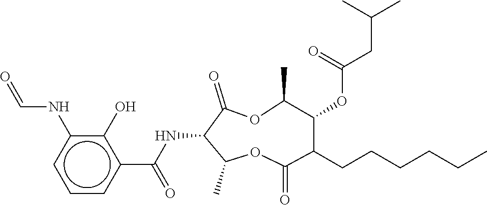 |
| Antimycin A3 | 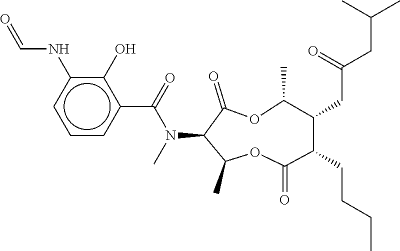 |
| AGN-194310 | 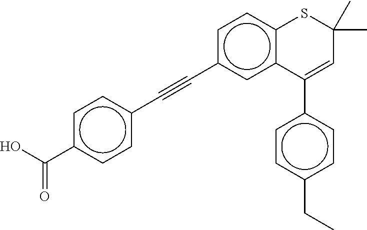 |
| TUBERCIDIN | 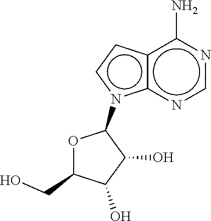 |
| ON-09310 | 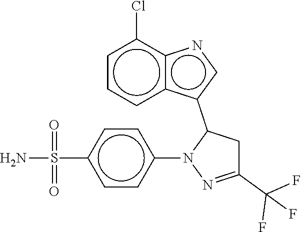 |
| tanaproget | 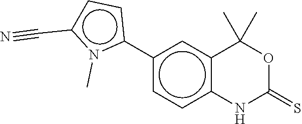 |
| auranofin | 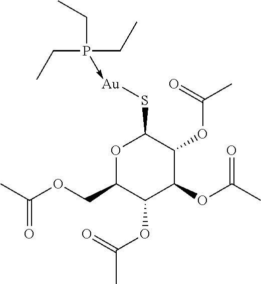 |
| Analog of OGT- 719 | 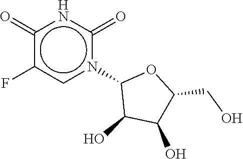 |
| Azathioprin | 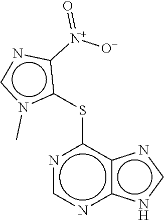 |
| Mercaptopurine | 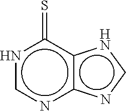 |
| FO 152 | 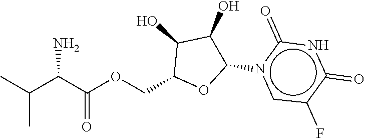 |
| Cloturin | 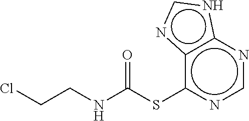 |
| Tioguanine | 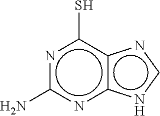 |
| Metildigoxin | 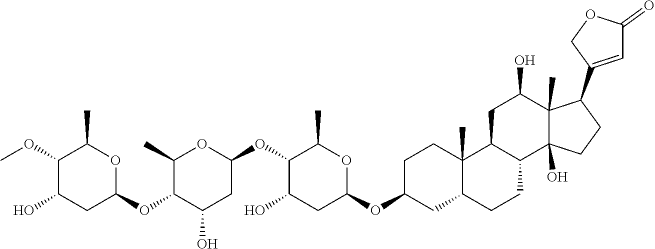 [SMILES: CO[C@H]1[C@@H](O)C[C@H](O[C@H]2[C@@H](O)C[C@H](O[C@H]3[C@@H](O)C[C@H](O[C@H]4CC[C@@]5(C)[C@H](CC[C@@H]6[C@@H]5C[C@@H](O)[C@]5(C)[C@@H](C7=CC(=O)OC7)CC[C@]65O)C4)O[C@@H]3C)O[C@@H]2C)O[C@@H]1C] |
| 6- Methylmercaptopurine riboside (metabolite) | 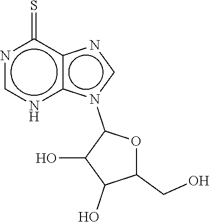 |
| BN-82685 | 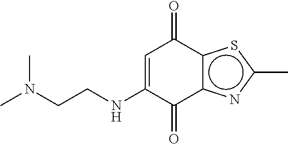 |
| Peruvoside (analog) | 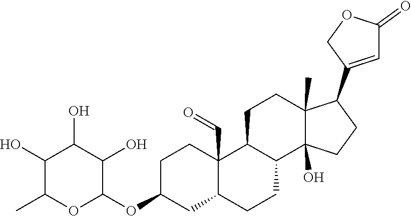 [SMILES: CC1OC(O[C@H]2CC[C@@]3(C=O)[C@H](CC[C@@H]4[C@@H]3CC[C@]3(C)[C@@H](C5=CC(=O)OC5)CC[C@]43O)C2)C(O)C(O)C1O] |
| ethinyl estradiol sulfonate | 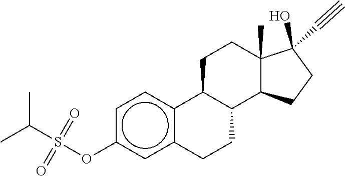 |
| Mavatrep | 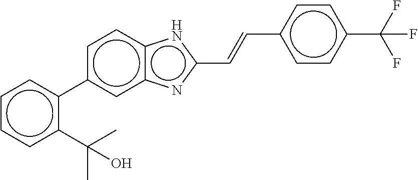 |
| ZD 2138 | 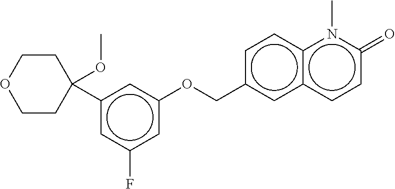 |
| TULOPAFANT | 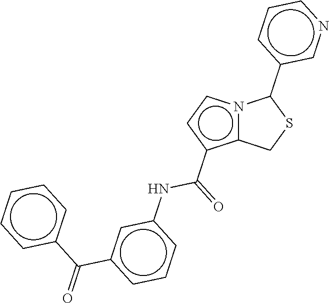 |
| oxametacin | 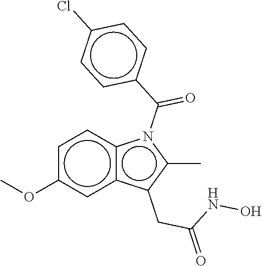 |
| LANATOSIDE B | 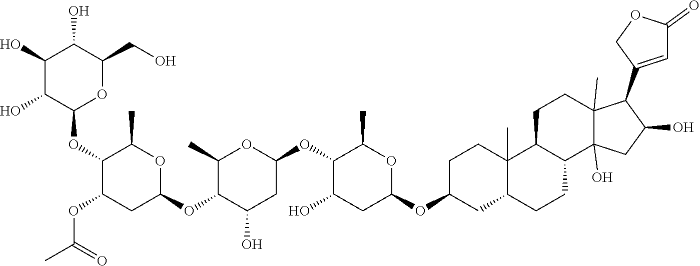 |
| INS-117548 | 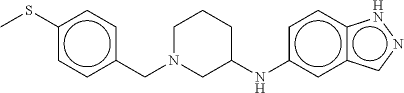 |
| SAR-407899 | 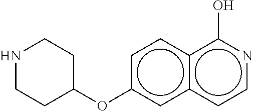 |
| GENZ-29155 | 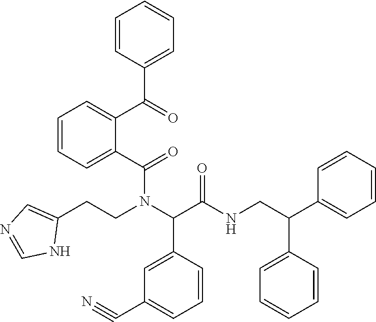 [SMILES: N#Cc1cccc(C(C(=O)NCC(c2ccccc2)c2ccccc2)N(CCc2cnc[nH]2)C(=O)c2ccccc2C(=O)c2ccccc2)c1] |
| Elubrixin tosylate | 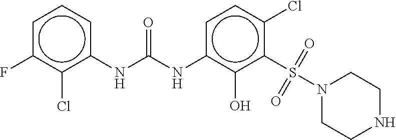 |

3. The method according to claim 1 or 2, wherein the pathogen is a coronavirus.

4. The method according to any one of claims 1 to 3, wherein the pathogen is severe acute respiratory syndrome coronavirus 2 (SARS-CoV-2).

5. The method according to any one of claims 1 to 4, further comprising administering an anti-infective agent.

6. The method according to claim 5, wherein the anti-infective agent comprises an anti-viral agent.

7. The method according to claim 6, wherein the anti-viral agent is selected from the group consisting of entry-inhibiting drugs, uncoating inhibiting drugs, reverse transcriptase inhibiting drugs, antisense drugs, ribozyme drugs, protease inhibitors, assembly inhibiting drugs, and release inhibiting drugs.

8. The method according to claim 5 or 6, wherein the anti-viral agent is selected from the group consisting of remdesivir, hydroxychloroquine, pyronaridine, azithromycin, and favipiravir.

9. The method according to claim 5 or 6, wherein the anti-viral agent is selected from the group consisting of amodiaquine, 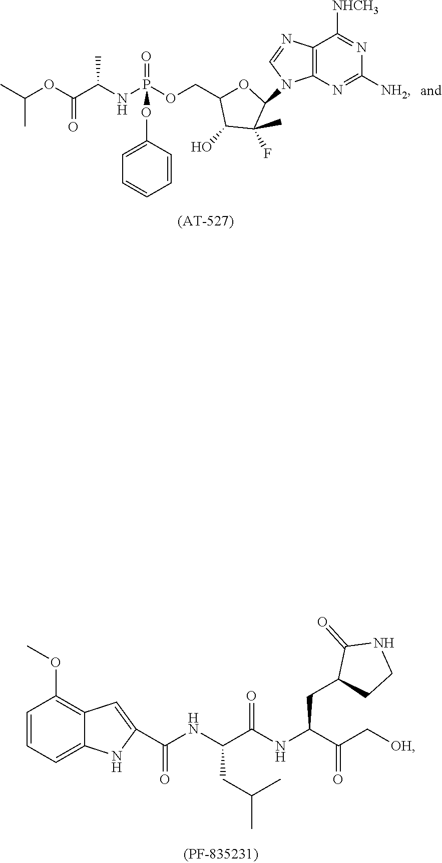 [SMILES: CNc1nc(N)nc2c1ncn2[C@@H]1OC(CO[P@@](=O)(N[C@@H](C)C(=O)OC(C)C)Oc2ccccc2)[C@@H](O)[C@@]1(C)F ; COc1cccc2[nH]c(C(=O)N[C@@H](CC(C)C)C(=O)N[C@@H](C[C@@H]3CCNC3=O)C(=O)CO)cc12]
optionally in combination with dexamethasone.

10. A compound according to formula RFM-011-200-5 or a pharmaceutically acceptable salt thereof:

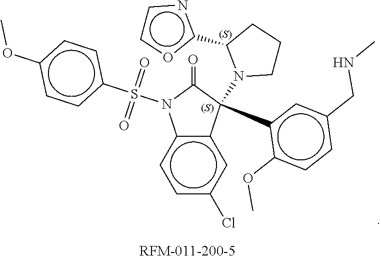

11. A compound according to formula RFM-007-454-4 or a pharmaceutically acceptable salt thereof: [chemical structure]
